(19) United States
(12) Patent Application Publication
(10) Pub. No.: US 2025/0375532 A1
(43) Pub. Date: Dec. 11, 2025

(54) DUAL AFFINITY NANOPARTICLES FOR THE TREATMENT OF CANCER

(71) Applicant: Vanderbilt University, Nashville, TN (US)

(72) Inventors: Michael R. KING, Nashville, TN (US); Maria LOPEZ CAVESTANY, Nashville, TN (US); Zhenjiang ZHANG, Nashville, TN (US)

(73) Assignee: Vanderbilt University, Nashville, TN (US)

(21) Appl. No.: 19/224,781

(22) Filed: May 31, 2025

Related U.S. Application Data
(60) Provisional application No. 63/656,326, filed on Jun. 5, 2024.

(51) Int. Cl. A61K 47/69; A61K 38/19; A61K 47/64; A61K 47/68; A61P 35/00; C07K 16/18

(52) U.S. Cl. CPC A61K 47/6913; A61K 38/19; A61K 47/6425; A61K 47/6843; A61P 35/00; C07K 16/18

(57) ABSTRACT

The present disclosure described bi-functional delivery particles with the ability to bind to two different cell types in a distinct fashion. Employing a first ligand with differential binding capabilities, the delivery particles may bind to a first target, such as cell, in a reversible fashion such that when they encounter a second target (e.g., cell) a second ligand that bind irreversibly to the second target will disrupt the binding to the first target. As such, the first target acts as a carrier to delivery the particle to a diagnostic or therapeutic second target. In particular aspects, the first and second cells are circulating cells.

### PRIORITY CLAIM

This application claims benefit of priority to U.S. Provisional Application Ser. No. 63/656,326, filed Jun. 5, 2024, the entire contents of which are hereby incorporated by reference.

### STATEMENT REGARDING FEDERALLY FUNDED RESEARCH

This invention was made with government support under grant no. CA203991 awarded by the National Institutes of Health. The government has certain rights in the invention.

### BACKGROUND

### 1. Field of the Disclosure

The present disclosure relates generally to the fields of medicine, cell biology, and oncology. More particular, the disclosure relates to dual affinity nanoparticles that bind to low affinity targets on a non-cancerous cell and high affinity targets on a cancer cell.

### 2. Background

The use of targeted nanoparticles as carriers for anti-cancer agents is a highly popular method as it allows more concentrated doses of therapeutics to reach the affected area (Gu et al., 2007). This also avoids many cytotoxic effects the chemotherapies have in healthy cells by decreasing the amount of the drug that reaches nonspecific targets (Yao et al., 2020). Although such approaches have been widely pursued, there are still many limitations that exist for all nanoparticle systems: the brevity of circulation time and the specificity of the targeting, for instance. Once a nanoparticle has entered the bloodstream, the body has a variety of mechanisms to clear them such as renal and hepatic clearance, and natural extravasation to other tissues (Longmire et al., 2008; Chinen et al., 2015 Poon et al., 2019). Additionally, as nanoparticles travel through the circulation, they encounter platelets, plasma proteins, coagulation factors, and blood cells, potentially leading to immune responses and particle breakdown. These problems are magnified when the target cell is freely floating in the bloodstream, such as a circulating tumor cell (CTC) in the case of cancer.

High patient CTC, and CTC cluster count, is known to correlate strongly with poor overall and progression-free survival in many cancer types (Sastre et al., 2012; Abdalla et al., 2021; Naito et al., 2012; Shishido et al., 2019; Du et al., 2020; Liu et al., 2020; Tsai et al., 2019; Ortiz-Otero et al., 2021). Tumors shed cancer cells up to a maximum of about 4×10⁶ cancer cells per gram of tumor into the circulation (Butler & Gullino, 1975). Many factors contribute to increasing CTC counts in patient blood. Some chemotherapies are known to have a destabilizing effect on the already leaky vasculature within a tumor which aids in the shedding of cancer cells (Ortiz-Otero et al., 2020a; Harris et al., 2018; Karagiannis et al., 2017). Another factor that contributes to increasing CTC counts in patients is surgery, where cancer cells are shed by the tumor as it is taken out by physicians (Marshall & King, 2016; Sawabata et al., 2016; Alieva et al., 2018; Ortiz-Otero et al., 2021). The great majority of cancer cells are not able to survive the forces and interactions in the circulation, yet it has been shown that very few CTCs are needed to successfully form a secondary metastatic site (Schuster et al., 2021; Yoshida et al., 1993; Perea Paizal et al., 2021). Therefore, eliminating CTCs before they are able to extravasate and form a metastatic site is an important treatment mechanism that needs to be further explored.

Tumor necrosis factor-related apoptosis-inducing ligand (TRAIL) is produced by natural killer (NK) cells and selectively kills cancer cells by binding to death receptors 4 & 5 to induce apoptosis (von Karstedt et al., 2017). Normal cells are partially protected against TRAIL by the expression of decoy receptors, and differential expression of intracellular proteins. A study published in 2013 by the inventors' laboratory demonstrated that fluid shear stress (FSS) sensitized cancer cells to TRAIL-mediated apoptosis (Mitchell & King, 2013). Over the past 10 years, the lab has developed various different variations of a nanoparticle system which aims to induce apoptosis in CTCs and cancer cells in the lymphatic and circulatory systems by exploiting this finding. Liposomes were first functionalized with TRAIL and E-selectin, which helped tether them to healthy leukocytes in the circulation (Mitchell et al., 2014; Greenlee et al., 2021). Leukocytes have naturally occurring E-selectin ligands (ESL) on their cell membrane, which allow them to adhere to the E-selectin(ES)—rich endothelial tissue of the circulatory system in order to extravasate and traffic to other parts of the body during the inflammatory cascade (Long et al., 2001; Wild et al., 2001). This allowed for increased circulation time in the blood and resulted in high levels of colorectal cancer cell apoptosis under physiological flow conditions. In vivo experiments using these liposomes were also highly successful in treating colorectal cancer. This nanoparticle design was also proven to have anti-cancer effects in both prostate and breast cancer in the following years (Ortiz-Otero et al., 2021; Jyotsana et al., 2019; Wayne et al., 2016). Two successful variations to the original liposomes were established afterwards. The first swapped out E-selectin for an anti-NK cell antibody in an effort to treat cells flowing through the lymphatic system (Mitchell & King, 2014; Chandrasekaran et al., 2016). The idea is similar: the liposomes would passively target the cancer cells by attaching them to the surface of an NK cell. The liposome successfully induced apoptosis in cancer cells and showed increased retention in the lymphatic system. The second variation focused back on the circulatory system, but instead of leukocytes, the liposomes were designed to attach to healthy platelets (Ortiz-Otero et al., 2018; Ortiz-Otero et al., 2020b). This was achieved by replacing the E-selectin with vWF-A₁, a physiological ligand for the GPIba receptor on the platelet surface which exhibits “selectin-like” kinetics. This design saw success in inducing apoptosis in CRC and breast cancer cells in physiological flow conditions. But despite these advances, there remains a need for further improvements in the targeting of functionalized nanoparticles in vivo.

### SUMMARY

Thus, in accordance with the present disclosure, there is provided a delivery particle comprising (a) an anti-cancer agent; (b) a first targeting agent that binds to a non-cancer cell surface moiety; and (c) a second targeting agent that binds to a cancer cell surface moiety, wherein the relative affinity of said first targeting agent to said second targeting agent is such that the second targeting agent binding with the cancer cell surface moiety will outcompete the first agent binding with the non-cancer cell surface moiety.

The relative affinities for said first targeting agent and said second targeting agent may be less than 100 pN in strength and at least 1000 pN in strength, respectively, such as about 30 pN and about 1000-2000 pN, respectively. The delivery particle may be a bead, a gold particle, a polymeric particle, a vesicle (e.g., nanovesicle), a liposome, a nanoparticle (e.g., lipid nanoparticle), a nanotube, a nanorod, a micelle, or a dendritic macromolecule. The lipid nanoparticle may comprise phosphatidyl choline, sphingomyelin and cholesterol. The second targeting agent may be an antibody to said cancer cell surface moiety. The cancer cell surface moiety may be vimentin or PSMA. The delivery particle may be a phase separated liposome comprising at least three lipid types, and as the anti-cancer agent may be linked to one of said at least three lipid types, and/or said first and/or second targeting agent is linked to one of said at least three lipid types.

The first targeting agent may be an antibody or receptor for said non-cancer cell surface moiety, such as a receptor for a moiety found on a white blood cell. The receptor for said non-cancer cell surface moiety may be E-selectin, P-selectin, L-selectin or vWF-A1. The anti-cancer agent may be located in an internal phase of said delivery particle, such as a chemotherapeutic agent, a radiotherapeutic agent or a toxin. The anti-cancer agent may be located on the surface of said delivery particle. The anti-cancer agent may be TRAIL or Fas ligand. The targeting agent may be E-selectin, the second targeting agent may be an antibody that binds to vimentin and the anti-cancer agent may be TRAIL. The one or more of said anti-cancer agent, said first targeting agent and said second targeting agent may be conjugated to the surface of said delivery particle using click chemistry. The cancer cell may be a circulating cancer cell. The non-cancer cell may be a circulating non-cancer cell.

Also provided is a pharmaceutical formulation comprising the delivery particle as defined in the preceding paragraph disposed in a pharmaceutically acceptable carrier or diluent.

In another embodiment, there is provided a delivery particle comprising (a) a detectable label; (b) a first targeting agent that binds to a non-cancer cell surface moiety in a reversible fashion; and (c) a second targeting agent that irreversibly binds to a cancer cell surface moiety. Also provided is a pharmaceutical formulation comprising this delivery particle disposed in a pharmaceutically acceptable carrier or diluent.

Further provided is a kit comprising a delivery particle as defined here.

In yet another embodiment, there is provided a method of targeting a circulating cancer cell in a subject comprising administering to said subject a delivery particle as defined herein or a pharmaceutical formulation comprising the same. In yet a further embodiment, there is provided a method of treating a subject with cancer comprising administering to said subject a delivery particle as defined herein or a pharmaceutical composition comprising the same.

The cancer or cancer cell of the methods may be recurrent and/or drug resistant, or may be selected from the group consisting of liver cancer, pancreatic cancer, lung cancer, head & neck cancer, oral cancer, esophageal cancer, stomach cancer, colon cancer, colorectal cancer, breast cancer, testicular cancer, ovarian cancer, uterine cancer, melanoma, or leukemia.

The delivery particle may be administered more than once, such as 2, 3, 4, 5, 6, 7, 8, 9, 10, 11, 12, 13, 14, 15, 20, 25 or 30 times. The subject may be treated over a period one week, two weeks, three weeks, four weeks, one month, two months, three months, four months, five months, six months, 9 months, 12 months, 1 year, 2 years, 3 years, 4 years, 5 years or 10 years. The subject may be treated with a second cancer therapy, such chemotherapy, immunotherapy, radiotherapy, gene therapy or surgery, or a chemical TRAIL sensitizer. The cancer cell may be a circulating cancer cell and/or the non-cancer cell may be a circulating non-cancer cell. The cancer cell may be a circulating cancer cell and the non-cancer cell may be a circulating non-cancer cell.

In an additional embodiment, there is provided a delivery particle comprising (a) a first targeting agent that binds to a first cell; and (b) a second targeting agent that binds to a second cell, wherein the relative affinity of said first targeting agent to said second targeting agent is such that the second targeting agent binding to the second cell surface moiety will outcompete the first agent binding to the first cell surface moiety, resulting in transfer of the delivery particle from the first cell to the second cell, within the dynamic environment of blood flow.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIG. 1. Schematic detailing the two-step mechanism of the DA nanoparticle system. Step 1 shows the loose binding of the liposomes to healthy leukocytes (carrier cells) circulating in the blood stream. Step 2 is the transfer of the nanoparticles from the carrier cell to the target cell (CTC). Lastly, step 3 is the delivery of the therapeutic agent to the target cell.

FIGS. 2A-C. Liposomal nanoparticle synthesis and functionalization. (FIG. 2A) Diagram depicting the steps to synthesize uniform liposomes and the functionalization click-chemistry. (FIG. 2B) Dynamic diameter measurements of each liposome group without conjugated TRAIL. (FIG. 2C) Dynamic diameter measurements of each liposome group with conjugated TRAIL.

FIGS. 3A-E. Dual affinity liposomes bind to PLB985 cells. (FIG. 3A) Quantification of cell-surface vimentin on PLB985 cells compared to an isotype control (statistical analysis: unpaired, nonparametric t-test). (FIG. 3B) Overview of the experimental design. (FIG. 3C) Flow cytometry plots and quantification of liposome binding to PLB985 cells under physiological FSS (statistical analysis: ordinary one-way ANOVA with Tukey multiple comparisons test). (FIG. 3D) Flow cytometry plots and quantification of liposome binding to PLB985 cells in static conditions compared to the FSS results (statistical analysis: two-way ANOVA with Tukey multiple comparisons test). (FIG. 3E) Confocal microscopy images of PLB985 cells with bound liposomes showing DAPI in blue and the liposomes in red (scale bar=20 μm). Graphs in this figure show mean±SD for an n=3, where *p<0.05, **p<0.01, ***p<0.001, and ****p<0.0001.

FIGS. 4A-E. Dual affinity liposomes bind to HCT116 cells. (FIG. 4A) Quantification of cell-surface vimentin on HCT116 cells compared to an isotype control (statistical analysis: unpaired, nonparametric t-test). (FIG. 4B) Overview of the experimental design. (FIG. 4C) Flow cytometry plots and quantification of liposome binding to HCT116 cells under physiological FSS (statistical analysis: ordinary one-way ANOVA with Tukey multiple comparisons test). (FIG. 4D) Flow cytometry plots and quantification of liposome binding to HCT116 cells in static conditions compared to the FSS results (statistical analysis: two-way ANOVA with Tukey multiple comparisons test). (FIG. 4E) Confocal microscopy images of HCT116 cells with bound liposomes showing DAPI in blue and the liposomes in red (scale bar=20 μm). Graphs in this figure show mean±SD for an n=3, where *p<0.05, **p<0.01, ***p<0.001, and ****p<0.0001.

FIGS. 5A-C. Dual affinity liposomes transfer from PLB985 cells to HCT116 cells under physiological FSS. (FIG. 5A) Overview of the experimental design. (FIG. 5B) Flow cytometry plots and quantification of liposome binding to HCT116 cells under physiological FSS (statistical analysis: ordinary one-way ANOVA with Tukey multiple comparisons test). (FIG. 5C) Flow cytometry plots and quantification of liposome binding to HCT116 cells in static conditions compared to the FSS results (statistical analysis: two-way ANOVA with Šídak's multiple comparisons test). Graphs in this figure show mean±SD for an n=3, where *p<0.05, **p<0.01, ***p<0.001, and ****p<0.0001.

FIGS. 6A-C. Quantification of liposome binding in static conditions via flow cytometry. (FIG. 6A) PLB985 cells; (FIG. 6B) HCT116 cells; (FIG. 6C) HCT116 cells after the transfer experiment with PL985 cells.

FIGS. 7A-B. Preparation and characterization of phase-separated GUVs and liposomes. (FIG. 7A) Typical confocal microscopy images of GUVs showing separation of phases (green=DSPC, re =DOPC) to different extent. Scale bars=10 μm. (FIG. 7B) Size distribution of naked liposomes and liposomes conjugated with varying numbers of TRAIL molecules.

FIGS. 8A-B. Apoptotic activity of TRAIL liposomes. (FIG. 8A) Representative flow cytometry plots of PC3 cells after treatment with TRAIL liposomes. Cell viability of PC3 cells and DU145 cells. (FIG. 8B) Incubation concentrations of TRAIL liposomes are 100 ng mL⁻¹ TRAIL for the treatment of PC3 and 250 ng mL⁻¹ TRAIL for DU145 cells. *p<0.05 and **p<0.01.

FIG. 9. Apoptotic activity of the TRAIL liposomes to Jurkat cells. TRAIL concentration was 50 ng mL⁻¹. * p<0.05 and ***p<0.001.

### DESCRIPTION OF ILLUSTRATIVE EMBODIMENTS

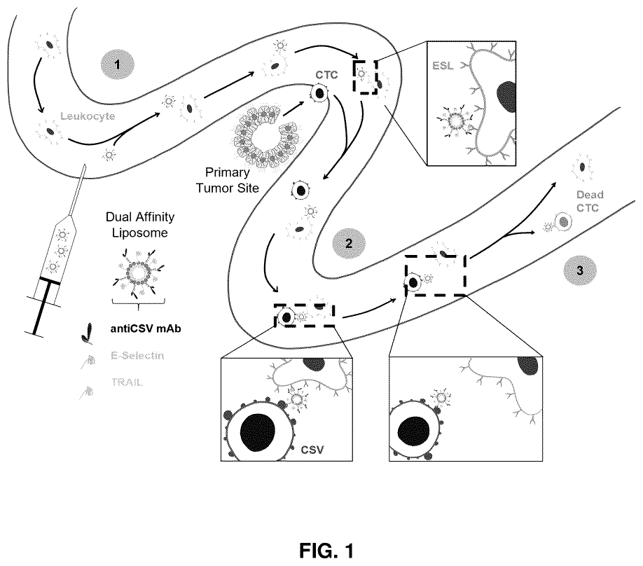

As discussed above, while nanoparticle therapeutics show great promise in the treatment of cancer, there remain challenges in deploying this technology as a bona fide cancer therapy. To that end, the inventors developed two-stage nanoparticle delivery platform relying on the dual functionalization of a liposome with moieties that have fundamentally different strengths of adhesion and binding kinetics to bind to a carrier cell (leukocyte) to the target cell (CTC) (FIG. 1). The dual affinity (DA) liposomes have TRAIL, E-selectin, and anti-cell surface vimentin (CSV) half antibodies on their surface. The first stage of the delivery mechanism is dependent on the ES to ESL catch-slip bond, which is known to be weak and reversible (Helms et al., 2016). The bond forms very quickly, within 0.5 to 16 seconds, and ruptures under an applied force over about 30 pN (Long et al., 2001; Snook & Guilford, 2010; Rocheleau et al., 2016). Through the use of E-selectin molecules on the liposome surface, one can take advantage of a healthy and abundant ligand on the cell surface of leukocytes, converting them to carrier cells in the bloodstream. Once the liposomes have entered the circulatory system, they will tether themselves to healthy leukocytes through the ES/ESL bond. This allows the nanoparticles to be dynamically transported and protected by the carrier cell until they have found their target. In this way, it allows them to circulate for longer periods of time in the bloodstream and evading the renal clearance and the natural extravasation of nanoparticles. The second stage of the system is triggered once the leukocyte-tethered liposomes come into contact with the CTC, they detach from the leukocyte surface and bind to the vimentin on the CTC surface. Antigen/antibody bonds are considered strong and irreversible, needing over 1000-2000 pN of force to rupture (Allen et al., 1997; Dammer et al., 1996). Once the liposome has been transferred from the surface of the leukocyte to that of the cancer cell, the therapeutic agent that it transports can take effect on the intended target. In this way, the CTC will be killed in the circulation, preventing the formation of metastatic lesions and advancement of cancer stage.

These and other aspects of the disclosure are described in detail below.

### I. Delivery Particles

The delivery particles of the disclosure can comprise a wide variety of platforms comprising beads, gold and polymeric particles, and (nano) vesicles. In particular, liposomes and lipid nanoparticles are contemplated. Liposomes are hollow spherical vesicles composed of lipids arranged in a similar fashion as those lipids which make up the cell membrane. They have, in another embodiment, an internal aqueous space for entrapping water-soluble compounds and range in size from 0.05 to several microns in diameter. When less than 1 micron in diameter, these can be considered a form of nanoparticle.

The term “lipid nanoparticle” refers to a particle having at least one dimension on the order of nanometers (e.g., 1-999 nm), which includes one or more lipids. In some embodiments, lipid nanoparticles are included in a formulation comprising one or more molecules as described herein. In some embodiments, such lipid nanoparticles comprise a cationic lipid and one or more excipients selected from neutral lipids, charged lipids, steroids and polymer conjugated lipids (e.g., a pegylated lipid such as a pegylated lipid of structure (IV), such as compound Iva). In some embodiments, one or more molecules are encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells, e.g., an adverse immune response. The one or more molecules may also be attached to the surface of the liposomes or LNPs.

In various embodiments, the lipid nanoparticles have a mean diameter from about 50 to about 500 nm, about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic. In some embodiments, the nucleoside-modified RNA, when present in the lipid nanoparticles, is resistant in aqueous solution to degradation with a nuclease.

The LNP may comprise any lipid capable of forming a particle to which the one or more molecules are attached, or in which the one or more molecules are encapsulated. The term “lipid” refers to a group of organic compounds that are derivatives of fatty acids (e.g., esters) and are generally characterized by being insoluble in water but soluble in many organic solvents. Lipids are usually divided in at least three classes: (1) “simple lipids” which include fats and oils as well as waxes; (2) “compound lipids” which include phospholipids and glycolipids; and (3) “derived lipids” such as steroids.

In one embodiment, the LNP comprises one or more cationic lipids, and one or more stabilizing lipids. Stabilizing lipids include neutral lipids and pegylated lipids.

In one embodiment, the LNP comprises a cationic lipid. As used herein, the term “cationic lipid” refers to a lipid that is cationic or becomes cationic (protonated) as the pH is lowered below the pK of the ionizable group of the lipid but is progressively more neutral at higher pH values. At pH values below the pK, the lipid is then able to associate with negatively charged nucleic acids. In some embodiments, the cationic lipid comprises a zwitterionic lipid that assumes a positive charge on pH decrease.

In some embodiments, the cationic lipid comprises any of a number of lipid species which carry a net positive charge at a selective pH, such as physiological pH. Such lipids include, but are not limited to, N,N-dioleyl-N,N-dimethylammonium chloride (DODAC); N-(2,3-dioleyloxy) propyl)-N,N,N-trimethylammonium chloride (DOTMA); N,N-distearyl-N,N-dimethylammonium bromide (DDAB); N-(2,3-dioleoyloxy)propyl)-N,N,N-trimethylammonium chloride (DOTAP); 3-(N-(N′,N′-dimethylaminoethane)-carbamoyl) cholesterol (DC-Chol), N-(1-(2,3-dioleoyloxy)propyl)-N-2-(sperminecarboxamido)ethyl)-N,N-dimethylammonium trifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy)propylamine (DODMA), and N-(1,2-dimyristyloxyprop-3-yl)-N,N-dimethyl-N-hydroxyethyl ammonium bromide (DMRIE).

Additionally, a number of commercial preparations of cationic lipids are available which can be used in the present disclosure. These include, for example, LIPOFECTIN® (commercially available cationic liposomes comprising DOTMA and 1,2-dioleoyl-sn-3-phosphoethanolamine (DOPE), from GIBCO/BRL, Grand Island, N.Y.); LIPOFECTAMINE® (commercially available cationic liposomes comprising N-(1-(2,3-dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.). The following lipids are cationic and have a positive charge at below physiological pH: DODAP, DODMA, DMDMA, 1,2-dilinoleyloxy-N,N-dimethylaminopropane (DLinDMA), 1,2-dilinolenyloxy-N,N-dimethylaminopropane (DLenDMA).

In one embodiment, the cationic lipid is an amino lipid. Suitable amino lipids useful in the disclosure include those described in WO 2012/016184, incorporated herein by reference in its entirety. Representative amino lipids include, but are not limited to, 1,2-dilinoleyoxy-3-(dimethylamino) acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino) propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N,N-dimethylamino) ethoxypropane (DLin-EG-DMA), and 2,2-dilinoleyl-4-dimethylaminomethyl-[1,3]-dioxolane (DLin-K-DMA).

In some embodiments, the LNP comprises one or more additional lipids which stabilize the formation of particles during their formation. Suitable stabilizing lipids include neutral lipids and anionic lipids. The term “neutral lipid” refers to any one of a number of lipid species that exist in either an uncharged or neutral zwitterionic form at physiological pH. Representative neutral lipids include diacylphosphatidylcholines, diacylphosphatidylethanolamines, ceramides, sphingomyelins, dihydro sphingomyelins, cephalins, and cerebrosides. Exemplary neutral lipids include, for example, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), and 1,2-dielaidoyl-sn-glycero-3-phophoethanolamine (transDOPE). In one embodiment, the neutral lipid is 1,2-distearoyl-sn-glycero-3-phosphocholine (DSPC).

In some embodiments, the LNPs comprise a neutral lipid selected from DSPC, DPPC, DMPC, DOPC, POPC, DOPE and SM. In various embodiments, the molar ratio of the cationic lipid (e.g., lipid of Formula (I)) to the neutral lipid ranges from about 2:1 to about 8:1. In various embodiments, the LNPs further comprise a steroid or steroid analogue.

In some embodiments, the steroid or steroid analogue is cholesterol. In some of these embodiments, the molar ratio of the cationic lipid to cholesterol ranges from about 2:1 to 1:1.

The term “anionic lipid” refers to any lipid that is negatively charged at physiological pH. These lipids include phosphatidylglycerol, cardiolipin, diacylphosphatidylserine, diacylphosphatidic acid, N-dodecanoylphosphatidylethanolamines, N-succinylphosphatidylethanolamines, N-glutarylphosphatidylethanolamines, lysylphosphatidylglycerols, palmitoyloleyolphosphatidylglycerol (POPG), and other anionic modifying groups joined to neutral lipids.

In some embodiments, the LNP comprises glycolipids (e.g., monosialoganglioside GM1). In some embodiments, the LNP comprises a sterol, such as cholesterol.

In some embodiments, the LNPs comprise a polymer conjugated lipid. The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-s-DMG) and the like.

In some embodiments, the LNP comprises an additional, stabilizing lipid which is a polyethylene glycol-lipid (pegylated lipid). Suitable polyethylene glycol-lipids include PEG-modified phosphatidylethanolamine, PEG-modified phosphatidic acid, PEG-modified ceramides (e.g., PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In one embodiment, the polyethylene glycol-lipid is N-[(methoxy poly(ethylene glycol)2000)carbamyl]-1,2-dimyristyloxlpropyl-3-amine (PEG-c-DMA). In one embodiment, the polyethylene glycol-lipid is PEG-c-DOMG). In other embodiments, the LNPs comprise a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG), a pegylated ceramide (PEG-5 cer), or a PEG dialkoxypropylcarbamate. In various embodiments, the molar ratio of the cationic lipid to the pegylated lipid ranges from about 100:1 to about 25:1.

In some embodiments, the LNPs comprise a pegylated lipid. Other exemplary LNPs and their manufacture are described in the art, for example in U.S. Patent Application Publication No. US20120276209, Semple et al., 2010, Nat Biotechnol., 28(2): 172-176; Akinc et al., 2010, Mol Ther., 18(7): 1357-1364; Basha et al., 2011, Mol Ther, 19(12): 2186-2200; Leung et al., 2012, J Phys Chem C Nanomater Interfaces, 116(34): 18440-18450; Lee et al., 2012, Int J Cancer., 131(5): E781-90; Belliveau et al., 2012, Mol Ther nucleic Acids, 1: e37; Jayaraman et al., 2012, Angew Chem Int Ed Engl., 51(34): 8529-8533; Mui et al., 2013, Mol Ther Nucleic Acids. 2, e139; Maier et al., 2013, Mol Ther., 21(8): 1570-1578; and Tam et al., 2013, Nanomedicine, 9(5): 665-74, each of which are incorporated by reference in their entirety.

### II. Delivery Particle Modification with Functional Features

In accordance with the present disclosure, the delivery particles described herein will be functionalized in three ways. First, the delivery particles will be engineered to contain an anti-cancer molecule. Second, the delivery particles will be functionalized with a first targeting agent that can target a non-cancer cell ligand that is found on non-cancer cells, particularly those that travel throughout a subject's body and that are not stationary. The design requires that this be a relatively low affinity interaction, i.e., reversible. And third, the delivery particles will be functionalized with a second targeting agent that can target a cancer cell-specific or -selective ligand that is found exclusively or preferentially on the surface of a cancer cell. The design requires that this be a relatively high affinity interaction, i.e., near or at non-reversibility, and/or substantially higher than that of the first targeting agent.

Rapid association and dissociation of load-bearing chemical bonds between endothelial P-selectin and neutrophil P-selectin glycoprotein ligand-1 (PSGL-1) is responsible for a slow rolling motion of neutrophils across the luminal vessel surface that has been well studied. Due to this important biological role for selectin-carbohydrate bond kinetics, several efforts have been made to experimentally characterize the lifetime of an individual selectin bond. The most common approach is to covalently attach or chemisorb purified selectin molecule onto a glass substrate at low density, and then introduce leukocytes in the well-defined flow of a parallel-plate flow chamber. The surface concentration of molecules is reduced to a low value that supports transient pauses as the cells flow across the reactive surface, suggesting that the observed interactions are mediated by a small number of bonds. For a single selectin bond, the duration of the cell pause is then equivalent to the bond lifetime. The force exerted by the fluid on the cell is known from fluid mechanics; however, determining the precise mechanical force experienced by the molecule requires complex analysis (King et al., 2005). Other natural adhesion proteins have been demonstrated to also possess “selectin-like” binding kinetics, which are characterized as exhibiting reversible, cell-rolling adhesion, very different from the slow-forming, irreversible kinetics of antibody-antigen bonds (Dogget et al., 2002).

In general, it is simply necessary that the first and second affinity interactions be such that the second will always outcompete the first, permitting transfer of the delivery particle from, e.g., a non-cancer cell to a cancer cell. The specific biophysics of selectin bonds can be expressed in terms of a “force loading rate” (see Tees et al., 2001), but in general the rupture force required to break a selectin bond is about 30 pN, while the rupture force required to break an antigen-antibody bond is between about 1000-2000 pN. A computational model that simulates collision forces between cells in the circulation to be within 150-250 pN is provided by Isfahani and Freund (2012). Thus, an essentially “irreversible” bond may be considered here as “a bond that has a rupture force which greatly exceeds collision forces that regularly occur in the circulation.” Other references describing selectin bonds are Snook and Guilford (2010) and Rocheleau et al. (2016). A similar reference for antibody bonds is Allen et al. (1997).

### A. Anti-Cancer Agents

A very wide range of anti-cancer agents is contemplated, but particularly those that are capable of delivering a lethal effect to the cancer cell at the surface, i.e., without being internalized. These ligands such as TRAIL and Fas-L and could also be antibodies to receptors on the cells, such as those categorized as immune checkpoint inhibitors and the death receptors. Studies have reported monoclonal antibodies that will crosslink and bind the death receptors to induce apoptosis in a manner similar to TRAIL. Radionuclides may also function external to the cancer cell, such as the recent Pluvicto drug to treat prostate cancer. Alternatively, the delivery agent may be used to transport an anti-cancer agent into a cancer cell so that the agent can engage an internal target, such as a chemotherapeutics, radiotherapeutics or toxins that target internal cancer cell molecules and pathways. Such agents and therapies are discussed below in the section dealing with combination treatments (where the agent/therapy is used in combination with a fully function delivery agents).

TRAIL. In the field of cell biology, TNF-related apoptosis-inducing ligand (TRAIL), is a protein functioning as a ligand that induces the process of cell death called apoptosis. TRAIL is a cytokine that is produced and secreted by most normal tissue cells. It causes apoptosis primarily in tumor cells, by binding to certain death receptors. TRAIL and its receptors have been used as the targets of several anti-cancer therapeutics since the mid-1990s, such as Mapatumumab. TRAIL has also been designated CD253 (cluster of differentiation 253) and TNFSF10 (tumor necrosis factor (ligand) superfamily, member 10). In humans, the gene that encodes TRAIL is located at chromosome 3q26, which is not close to other TNF family members. The genomic structure of the TRAIL gene spans approximately 20 kb and is composed of five exonic segments 222, 138, 42, 106, and 1245 nucleotides and four introns of approximately 8.2, 3.2, 2.3 and 2.3 kb. The TRAIL gene lacks TATA and CAAT boxes and the promoter region contains putative response elements for transcription factors GATA, AP-1, C/EBP, SP-1, OCT-1, AP3, PEA3, CF-1, and ISRE. TIC10 (which causes expression of TRAIL) was investigated in mice with various tumor types. Small molecule ONC201 causes expression of TRAIL which kills some cancer cells.

TRAIL shows homology to other members of the tumor necrosis factor superfamily. It is composed of 281 amino acids and has characteristics of a type II transmembrane protein. The N-terminal cytoplasmic domain is not conserved across family members; however, the C-terminal extracellular domain is conserved and can be proteolytically cleaved from the cell surface. TRAIL forms a homotrimer that binds three receptor molecules.

TRAIL binds to the death receptors DR4 (TRAIL-RI) and DR5 (TRAIL-RII). The process of apoptosis is caspase-8-dependent. Caspase-8 activates downstream effector caspases including procaspase-3,-6, and -7, leading to activation of specific kinases. TRAIL also binds the receptors DcR1 and DcR2, which do not contain a cytoplasmic domain (DcR1) or contain a truncated death domain (DcR2). DcR1 functions as a TRAIL-neutralizing decoy-receptor. The cytoplasmic domain of DcR2 is functional and activates NFkappaB. In cells expressing DcR2, TRAIL binding therefore activates NFkappaB, leading to transcription of genes known to antagonize the death signaling pathway and/or to promote inflammation. Application of engineered ligands that have variable affinity for different death (DR4 and DR5) and decoy receptors (DCR1 and DCR2) may allow selective targeting of cancer cells by controlling activation of Type 1/Type 2 pathways of cell death and single cell fluctuations. Luminescent iridium complex-peptide hybrids, which mimic TRAIL, have recently been synthesized in vitro. These artificial TRAIL mimics bind to DR4/DR5 on cancer cells and induce cell death via both apoptosis and necrosis, which makes them a potential candidate for anticancer drug development.

FasL. Fas ligand (FasL or CD95L or CD178) is a type-II transmembrane protein expressed on cytotoxic T lymphocytes and natural killer (NK) cells. Its binding with Fas receptor (FasR) induces programmed cell death in the FasR-carrying target cell. Fas ligand/receptor interactions play an important role in the regulation of the immune system and the progression of cancer.

### B. Cancer Cell Targeting Agent

In general, the targeting agent for such markers will typically be an antibody or antigen-binding fragment thereof. The basic four-chain antibody unit is a heterotetrameric glycoprotein composed of two identical light (L) chains and two identical heavy (H) chains. An IgM antibody consists of 5 basic heterotetramer units along with an additional polypeptide called J chain, and therefore contain 10 antigen binding sites, while secreted IgA antibodies can polymerize to form polyvalent assemblages comprising 2-5 of the basic 4-chain units along with J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable region (V_{H}) followed by three constant domains (C_{H}) for each of the alpha and gamma chains and four C_{H} domains for mu and isotypes. Each L chain has at the N-terminus, a variable region (V_{L}) followed by a constant domain (C_{L}) at its other end. The V_{L} is aligned with the V_{H} and the C_{L} is aligned with the first constant domain of the heavy chain (C_{H1}). Particular amino acid residues are believed to form an interface between the light chain and heavy chain variable regions. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71, and Chapter 6.

The L chain from any vertebrate species can be assigned to one of two clearly distinct types, called kappa and lambda based on the amino acid sequences of their constant domains (C_{L}). Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated alpha, delta, epsilon, gamma and mu, respectively. They gamma and alpha classes are further divided into subclasses on the basis of relatively minor differences in C_{H} sequence and function, humans express the following subclasses: IgG1, IgG2, IgG3, IgG4, IgA1, and IgA2.

The term “variable” refers to the fact that certain segments of the V domains differ extensively in sequence among antibodies. The V domain mediates antigen binding and defines specificity of a particular antibody for its particular antigen. However, the variability is not evenly distributed across the 110-amino acid span of the variable regions. Instead, the V regions consist of relatively invariant stretches called framework regions (FRs) of 15-30 amino acids separated by shorter regions of extreme variability called “hypervariable regions” that are each 9-12 amino acids long. The variable regions of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC), antibody-dependent cellular phagocytosis (ADCP), antibody-dependent neutrophil phagocytosis (ADNP), and antibody-dependent complement deposition (ADCD).

The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35 (H1), 50-65 (H2) and 95-102 (H3) in the V_{H} when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the V_{L}, and 26-32 (H1), 52-56 (H2) and 95-101 (H3) in the V_{H} when numbered in accordance with the Chothia numbering system; Chothia and Lesk, J. Mol. Biol. 196: 901-917 (1987)); and/or those residues from a “hypervariable loop”/CDR (e.g., residues 27-38 (L1), 56-65 (L2) and 105-120 (L3) in the V_{L}, and 27-38 (H1), 56-65 (H2) and 105-120 (H3) in the V_{H} when numbered in accordance with the IMGT numbering system; Lefranc, M. P. et al. Nucl. Acids Res. 27: 209-212 (1999), Ruiz, M. et al. Nucl. Acids Res. 28219-221 (2000)). Optionally the antibody has symmetrical insertions at one or more of the following points 28, 36 (L1), 63, 74-75 (L2) and 123 (L3) in the V_{L}, and 28, 36 (H1), 63, 74-75 (H2) and 123 (H3) in the V_{sub}H when numbered in accordance with AHo; Honneger, A. and Plunkthun, A. J. Mol. Biol. 309: 657-670 (2001)).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present disclosure may be prepared by the hybridoma methodology first described by Kohler et al., Nature, 256: 495 (1975), or may be made using recombinant DNA methods in bacterial, eukaryotic animal or plant cells (see, e.g., U.S. Pat. No. 4,816,567) after single cell sorting of an antigen specific B cell, an antigen specific plasmablast responding to an infection or immunization, or capture of linked heavy and light chains from single cells in a bulk sorted antigen specific collection. The “monoclonal antibodies” may also be isolated from phage antibody libraries using the techniques described in Clackson et al., Nature, 352: 624-628 (1991) and Marks et al., J. Mol. Biol., 222: 581-597 (1991), for example.

Binding agent will also comprise antibody fragments (such as F(ab′), F(ab′)₂) that are produced, for example, by the proteolytic cleavage of the mAbs, or single-chain immunoglobulins producible, for example, via recombinant means. F(ab′) antibody derivatives are monovalent, while F(ab′)₂ antibody derivatives are bivalent. In one embodiment, such fragments can be combined with one another, or with other antibody fragments or receptor ligands to form “chimeric” binding molecules. Significantly, such chimeric molecules may contain substituents capable of binding to different epitopes of the same molecule.

A variety of different cancer cell markers may be employed according to the present disclosure. Ideally, the cancer marker would reside on the surface of circulating tumor cells and would be overexpressed in cancer but show minimal to no surface expression on blood cells or endothelial cells. One example of this, in the case of prostate cancer, is PSMA, which is typically only expressed on the surface of cells originating from the prostate in men.

Vimentin. Vimentin is a structural protein that in humans is encoded by the VIM gene. Vimentin is a type III intermediate filament (IF) protein that is expressed in mesenchymal cells. IF proteins are found in all animal cells as well as bacteria. Intermediate filaments, along with tubulin-based microtubules and actin based microfilaments, comprise the cytoskeleton. All IF proteins are expressed in a highly developmentally regulated fashion; vimentin is the major cytoskeletal component of mesenchymal cells. Because of this, vimentin is often used as a marker of mesenchymally-derived cells or cells undergoing an epithelial-to-mesenchymal transition (EMT) during both normal development and metastatic progression. In particular, the present disclosure will employ cell surface vimentin (CSV) as opposed to cytoplasmic vimentin.

A vimentin monomer, like all other intermediate filaments, has a central α-helical domain, capped on each end by non-helical amino (head) and carboxyl (tail) domains. Two monomers are likely co-translationally expressed in a way that facilitates their formation of a coiled-coil dimer, which is the basic subunit of vimentin assembly.

The α-helical sequences contain a pattern of hydrophobic amino acids that contribute to forming a “hydrophobic seal” on the surface of the helix. In addition, there is a periodic distribution of acidic and basic amino acids that seems to play an important role in stabilizing coiled-coil dimers. The spacing of the charged residues is optimal for ionic salt bridges, which allows for the stabilization of the α-helix structure. While this type of stabilization is intuitive for intrachain interactions, rather than interchain interactions, scientists have proposed that perhaps the switch from intrachain salt bridges formed by acidic and basic residues to the interchain ionic associations contributes to the assembly of the filament.

Vimentin plays a significant role in supporting and anchoring the position of the organelles in the cytosol. Vimentin is attached to the nucleus, endoplasmic reticulum, and mitochondria, either laterally or terminally.

The dynamic nature of vimentin is important when offering flexibility to the cell. Scientists found that vimentin provided cells with a resilience absent from the microtubule or actin filament networks, when under mechanical stress in vivo. Therefore, in general, it is accepted that vimentin is the cytoskeletal component responsible for maintaining cell integrity. (It was found that cells without vimentin are extremely delicate when disturbed with a micropuncture). Transgenic mice that lack vimentin appeared normal and did not show functional differences. It is possible that the microtubule network may have compensated for the absence of the intermediate network. This result supports an intimate interaction between microtubules and vimentin. Moreover, when microtubule depolymerizers were present, vimentin reorganization occurred, once again implying a relationship between the two systems. On the other hand, wounded mice that lack the vimentin gene heal slower than their wild-type counterparts. In essence, vimentin is responsible for maintaining cell shape, integrity of the cytoplasm, and stabilizing cytoskeletal interactions. Vimentin has been shown to eliminate toxic proteins in JUNQ and IPOD inclusion bodies in asymmetric division of mammalian cell lines.

Also, vimentin is found to control the transport of low-density lipoprotein-derived cholesterol from a lysosome to the site of esterification. With the blocking of transport of LDL-derived cholesterol inside the cell, cells were found to store a much lower percentage of the lipoprotein than normal cells with vimentin. This dependence seems to be the first process of a biochemical function in any cell that depends on a cellular intermediate filament network. This type of dependence has ramifications on the adrenal cells, which rely on cholesteryl esters derived from LDL. Vimentin plays a role in aggresome formation, where it forms a cage surrounding a core of aggregated protein.

Vimentin has been shown to interact with DSP, MEN1, MYST2, PKN1, PRKCI, PLEC, SPTAN1, UPP1, and YWHAZ. The 3′ UTR of Vimentin mRNA has been found to bind a 46 kDa protein.

### C. Non-Cancer Targeting Agent

A variety of different non-cancer cell markers may be employed according to the present disclosure. Some of these are discussed below.

Selectins. E-selectin. E-selectin, also known as CD62 antigen-like family member E (CD62E), endothelial-leukocyte adhesion molecule 1 (ELAM-1), or leukocyte-endothelial cell adhesion molecule 2 (LECAM2), is a selectin cell adhesion molecule expressed only on endothelial cells activated by cytokines. Like other selectins, it plays an important part in inflammation. In humans, E-selectin is encoded by the SELE gene.

E-selectin has a cassette structure: an N-terminal, C-type lectin domain, an EGF (epidermal-growth-factor)-like domain, 6 Sushi domain (SCR repeat) units, a transmembrane domain (TM) and an intracellular cytoplasmic tail (cyto). The three-dimensional structure of the ligand-binding region of human E-selectin has been determined at 2.0 Å resolution in 1994. The structure reveals limited contact between the two domains and a coordination of Ca²⁺ not predicted from other C-type lectins. Structure/function analysis indicates a defined region and specific amino-acid side chains that may be involved in ligand binding. The E-selectin bound to sialyl-Lewis^{X} (SLe^{X}; NeuNAcα2,3Galβ1,4[Fucα1,3]GlcNAc) tetrasaccharide was solved in 2000.

In humans, E-selectin is encoded by the SELE gene. Its C-type lectin domain, EGF-like, SCR repeats, and transmembrane domains are each encoded by separate exons, whereas the E-selectin cytosolic domain derives from two exons. The E-selectin locus flanks the L-selectin locus on chromosome 1.

Different from P-selectin, which is stored in vesicles called Weibel-Palade bodies, E-selectin is not stored in the cell and has to be transcribed, translated, and transported to the cell surface. The production of E-selectin is stimulated by the expression of P-selectin which in turn, is stimulated by tumor necrosis factor α (TNFα), interleukin-1 (IL-1) and lipopolysaccharide (LPS). It takes about two hours, after cytokine recognition, for E-selectin to be expressed on the endothelial cell's surface. Maximal expression of E-selectin occurs around 6-12 hours after cytokine stimulation, and levels return to baseline within 24 hours.

Shear forces are also found to affect E-selectin expression. A high laminar shear enhances acute endothelial cell response to interleukin-1β in naïve or shear-conditioned endothelial cells as may be found in the pathological setting of ischemia/reperfusion injury while conferring rapid E-selectin down regulation to protect against chronic inflammation.

E-selectin recognizes and binds to sialylated carbohydrates present on the surface proteins of certain leukocytes. E-selectin ligands are expressed by neutrophils, monocytes, eosinophils, memory-effector T-like lymphocytes, and natural killer cells. Each of these cell types is found in acute and chronic inflammatory sites in association with expression of E-selectin, thus implicating E-selectin in the recruitment of these cells to such inflammatory sites. These carbohydrates include members of the Lewis X and Lewis A families found on monocytes, granulocytes, and T-lymphocytes.

The glycoprotein ESL-1, present on neutrophils and myeloid cells, was the first counter-receptor for E-selectin to be described. It is a variant of the tyrosine kinase FGF glycoreceptor, raising the possibility that its binding to E-selectin is involved in initiating signaling in the bound cells. P-selectin glycoprotein ligand-1 (PSGL-1) derived from human neutrophils is also a high-efficiency ligand for endothelium-expressed E-selectin under flow. It mediates the rolling of leukocytes on the activated endothelium surrounding an inflamed tissue. Both ESL-1 and PSGL-1 should bear sialyl Lewis a/x in order to bind E/P-selectins.

E-selectin is found to mediate the adhesion of tumor cells to endothelial cells, by binding to E-selectin ligands on the tumor cells. E-selectin ligands also play a role in cancer metastasis. The role of these two E-selectin ligands in metastasis in vivo is poorly defined and remains to be firmly demonstrated. PSGL-1 was detected on the surfaces of bone-metastatic prostate tumor cells, suggesting that it may have a functional role in the bone tropism of prostate tumor cells. In cancer cells, CD44, death receptor-3 (DR3), LAMP1, and LAMP2 were identified as E-selectin ligands present on colon cancer cells, and CD44v, Mac2-BP, and gangliosides were identified as E-selectin ligands present on breast cancer cells.

On human neutrophils the glycosphingolipid NeuAcα2-3Galβ1-4GlcNAcβ1-3[Galβ1-4(Fucα1-3) GlcNAcβ1-3]2[Galβ1-4GlcNAcβ1-3]2Galβ1-4GlcβCer (and closely related structures) are functional E-selectin receptors.

P-selectin. P-selectin is a type-1 transmembrane protein that in humans is encoded by the SELP gene. P-selectin functions as a cell adhesion molecule (CAM) on the surfaces of activated endothelial cells, which line the inner surface of blood vessels, and activated platelets. In unactivated endothelial cells, it is stored in granules called Weibel-Palade bodies. In unactivated platelets P-selectin is stored in α-granules. Other names for P-selectin include CD62P, Granule Membrane Protein 140 (GMP-140), and Platelet Activation-Dependent Granule to External Membrane Protein (PADGEM). It was first identified in endothelial cells in 1989.

P-selectin is located on chromosome 1q21-q24, spans>50 kb and contains 17 exons in humans. P-selectin is constitutively expressed in megakaryocytes (the precursor of platelets) and endothelial cells. P-selectin expression is induced by two distinct mechanisms. First, P-selectin is synthesized by megakaryocytes and endothelial cells, where it is sorted into the membranes of secretory granules. When megakaryocytes and endothelial cells are activated by agonists such as thrombin, P-selectin is rapidly translocated to the plasma membrane from granules. Secondly, increased levels of P-selectin mRNA and protein are induced by inflammatory mediators such as tumor necrosis factor-a (TNF-a), LPS, and interleukin-4 (IL-4). Although TNF-a and LPS increase levels of both mRNA and protein in murine models, they do not appear to affect mRNA in human endothelial cells, while IL-4 increases P-selectin transcription in both species. The elevated synthesis of P-selectin may play an important role in the delivery of protein to the cell surface. In ischemic stroke patients, plasma P-selectin concentration was reported to be highly correlated to plasminogen activator inhibitor-1 activity and tissue plasminogen activator activity.

P-selectin is found in endothelial cells and platelets where it is stored in Weibel-Palade bodies and α-granules, respectively. In response to inflammatory cytokines such as IL-4 and IL-13, P-selectin is translocated to the plasma membrane in endothelial cells. The extracellular region of P-selectin is composed of three different domains like other selectin types; a C-type lectin-like domain in the N-terminus, an EGF-like domain and a complement-binding protein-like domains (same as complement regulatory proteins: CRP) having short consensus repeats (˜60 amino acids). The number of CRP repeats is the major feature differentiating the type of selectin in extracellular region. In humans, P-selectin has nine repeats while E-selectin contains six and L-selectin has only two. P-selectin is anchored in transmembrane region that is followed by a short cytoplasmic tail region.

The primary ligand for P-selectin is P-selectin glycoprotein ligand-1 (PSGL-1) which is expressed on almost all leukocytes, although P-selectin also binds to heparan sulfate and fucoidans. PSGL-1 is situated on various hematopoietic cells such as neutrophils, eosinophils, lymphocytes, and monocytes, in which it mediates tethering and adhesion of these cells. However, PSGL-1 is not specific for P-selectin, as it can also function as a ligand for both E- and L-selectin.

P-selectin plays an essential role in the initial recruitment of leukocytes (white blood cells) to the site of injury during inflammation. When endothelial cells are activated by molecules such as histamine or thrombin during inflammation, P-selectin moves from an internal cell location to the endothelial cell surface.

Thrombin is one trigger which can stimulate endothelial-cell release of P-selectin and recent studies suggest an additional Ca²⁺-independent pathway involved in the release of P-selectin. Ligands for P-selectin on eosinophils and neutrophils are similar sialylated, protease-sensitive, endo-beta-galactosidase-resistant structures, clearly different from those reported for E-selectin, and suggest disparate roles for P-selectin and E-selectin during recruitment during inflammatory responses.

P-selectin is also very important in the recruitment and aggregation of platelets at areas of vascular injury. In a quiescent platelet, P-selectin is located on the inner wall of α-granules. Platelet activation (through agonists such as thrombin, Type II collagen and ADP) results in “membrane flipping” where the platelet releases α- and dense granules and the inner walls of the granules are exposed on the outside of the cell. The P-selectin then promotes platelet aggregation through platelet-fibrin and platelet-platelet binding.

P-selectin attaches to the actin cytoskeleton through anchor proteins that are still poorly characterized.

P-selectin has a functional role in tumor metastasis similar to E-selectin. P-selectin is expressed on the surface of both stimulated endothelial cells and activated platelets and helps cancer cells invade into the bloodstream for metastasis and provides local multiple growth factors, respectively. Moreover, platelets facilitate tumor metastasis by forming complexes with tumour cells and leukocytes in the vasculature, thus preventing recognition by macrophages. This is thought to contribute to the seeding of tumor microemboli in distant organs. In vivo mouse experiments have shown that a reduction in circulating platelets could reduce cancer metastasis.

The oligosaccharide sialylated Lewis x (sLe(x)) is expressed on the surface of tumor cells and can be recognized by E-selectin and P-selectin, playing on a key role in metastasis of the tumor. However, in the 4T1 breast cancer cell line, E-selectin reactivity is sLe(x) dependent while P-selectin reactivity is sLe(x)-independent, suggesting P-selectin binding is Ca²⁺-independent and sulfation-dependent. One of the sulfated ligands is chondroitin sulfate, a type of glycosaminoglycan (GAG). Its activity in tumor metastasis has been probed by the addition of heparin that functions to blocks tumor metastasis. In addition to GAGs, mucin is of interest in P-selectin mediated tumor metastasis. Selective removal of mucin results in reduced interaction between P-selectin and platelets in vivo and in vitro.

Heparin has long been known to represent anti-heparanase activity that is to keep an endoglycosidase from degrading heparan sulfate, one of the glycosaminoglycans, and to effectively inhibit P-selectin. Despite a striking effect of heparin on tumor progression shown in a number of clinical trials, the use of heparin as anti-cancer agent is limited because of its risk, which might induce adverse bleeding complications. Given those reasons, development of new compounds that target P-selectin is now emerging for cancer therapy. Among them, the inhibitory activity of semisynthetic sulfated tri mannose C-C-linked dimers (STMCs) to P-selectin was shown by the attenuation of tumor metastasis in vivo animal model, indicating the inhibition of interaction between tumor cell and endothelial cell is significant for blocking tumor dissemination.

Crizanlizumab is a monoclonal antibody against P-selectin which has now been approved by Novartis on Nov. 15, 2019 for the indication of vaso-occlusive crisis in sickle cell patients.

L-selectin. L-selectin, also known as CD62L, is a cell adhesion molecule found on the cell surface of leukocytes, and the blastocyst. It is coded for humans by the SELL gene. L-selectin belongs to the selectin family of proteins, which recognize sialylated carbohydrate groups containing a Sialyl LewisX (sLeX) determinant. L-selectin plays an important role in both the innate and adaptive immune responses by facilitating leukocyte-endothelial cell adhesion events. These tethering interactions are essential for the trafficking of monocytes and neutrophils into inflamed tissue as well as the homing of lymphocytes to secondary lymphoid organs. L-selectin is also expressed by lymphoid primed hematopoietic stem cells and may participate in the migration of these stem cells to the primary lymphoid organs. In addition to its function in the immune response, L-selectin is expressed on embryonic cells and facilitates the attachment of the blastocyst to the endometrial endothelium during human embryo implantation.

L-selectin is composed of multiple structural regions: an N-terminus C-type lectin domain, an adjacent epidermal growth factor-like domain, two to the consensus repeat units homologous to those found in C3/C4-binding proteins, an extracellular cleavage site, a short transmembrane domain, and a cytoplasmic tail. It is cleaved by ADAM17.

The nature of the interactions between L-selectin and ligand depends on many circumstances, primarily the location of anatomically defined sites in the high vessel venules (perivascular, extravascular and intravascular).

Because of the diversity of L-selectin ligands, signals that propagate downstream of L-selectin provide information about the position of the leukocyte within the multistep adhesion cascade (binding, rolling, adhesion, and transmigration).

While L-selectin ligands on the apical side of the endothelium have long been characterized as receptors for binding and rolling, glycans enriched on the basolateral side and in the basement membrane likely control quite different signals. The binding lifetime of L-selectin with apical ligands will be on the order of milliseconds, so in contrast, L-selectin-dependent adhesion in a microenvironment without hydrodynamic shear stress (e.g., within transmigrating pseudopods) will take seconds to minutes.

GlyCAM-1 is found in the high endothelial venules of the lymph nodes. CD34 is found on endothelial cells. MadCAM-1 is found on endothelial cells of gut-associated lymphoid tissue. PSGL-1 binds with low affinity.

L-selectin is expressed constitutively on most circulating leukocytes. Over time, these molecules are released through the process of ectodomain shedding and are replaced by newly synthesized L-selectin proteins. Ectodomain shedding is largely accomplished through cleavage by ADAM17. The human L-selectin gene (sell) is located on the long arm of chromosome 1 (1q24.2), and is arranged in tandem with its family members (in the order: L-, P-, and E-selectin). Human L-selectin consists of 10 exons and its transcription factor is FOXO 1, (Shishido et al., 2019) on the other hand the mouse sell gene is composed of 9 exons.

Subsequent splicing of exons into mature mRNA translates to a protein product with a predicted molecular mass of 30 kDa. L-selectin varies between cell types, has ranging molecular weight from 65 kDa in lymphocytes to 100 kDa in neutrophils, and is due to cell type-specific glycosylation. Most glycoproteins undergo either N- or O-linked glycosylation, and it is very likely that the type of L-selectin glycosylation determines the specific functions of individual cells, but this has not yet been investigated in detail.

L-selectin is expressed on naive T cells and is rapidly shed following T cell priming. L-selectin expression is re-activated in cytotoxic T cells once they exit the lymph node. Mature central memory T cells express L-selectin while effector memory cells do not. L-selectin is also expressed by naive B cells, with the loss of L-selectin distinguishing activated B cells destined to differentiate to antibody-secreting cells.

L-selectin is expressed on circulating neutrophils and is shed following neutrophil priming. Expression of L-selectin in neutrophils decreases with neutrophil aging. Classical monocytes express high levels of L-selectin while in circulation. Shedding of L-selectin from monocytes occurs during trans-endothelial migration.

L-selectin expression is also observed on oocytes and early-stage embryos. Blastocysts express L-selectin following, but not prior to emergence from the zona pellucida. An increase in L-selectin expression is observed when both the blastocyst and cytotrophoblast attach to the endometrium. L-selectin expression decreases by the 17th week of pregnancy, and remains low or non-existent until term (2017).

L-selectin acts as a “homing receptor” for lymphocytes to enter secondary lymphoid tissues via high endothelial venules. Ligands present on endothelial cells will bind to lymphocytes expressing L-selectin, slowing lymphocyte trafficking through the blood, and facilitating entry into a secondary lymphoid organ at that point. The receptor is commonly found on the cell surfaces of T cells. Naive T-lymphocytes, which have not yet encountered their specific antigen, need to enter secondary lymph nodes to encounter their antigen. Central memory T-lymphocytes, which have encountered antigen, express L-selectin to localize in secondary lymphoid organs. Here they reside ready to proliferate upon re-encountering antigen. Effector memory T-lymphocytes do not express L-selectin, as they circulate in the periphery and have immediate effector functions upon encountering antigen. High expression of L-selectin on human bone marrow progenitor cells is an early sign of cells becoming committed to lymphoid differentiation.

Similar to its role in homing lymphocytes to secondary lymphoid tissues, L-selectin expressed on the surface of monocytes and neutrophils is essential for facilitating the first stage of adhesion to venule epithelial cells (known as the “rolling stage”). Adhesion to activated epithelial cells is a critical step in the immune response as it allows these immune cells to emigrate from the bloodstream into inflamed tissue. Prolonged rolling and transmigration of neutrophils can trigger shedding of L-selectin from the neutrophil plasma membrane. The membrane-bound fragment left behind following cleavage of L-selectin has also been suggested to play a critical role in the interstitial chemotaxis of neutrophils along a cytokine gradient. L-selectin on neutrophils can result in its own ectodomain shedding, driven by activation of p38 MAPK followed by antibody-mediated clustering (AMC), after which L-selectin can behave as a cell adhesion molecule and signaling receptor. L-selectin shedding is not strictly consequence of neutrohpil transmigration, because it was observed that there is differences between neutrophil migration toward acute or chronic inflammation could differ in the expression and turnover of adhesion molecules.

L-selectin shedding also occurs in monocytes; however, in these cells shedding is triggered only during trans-endothelial and not by earlier stages of the adhesion process. The specific shedding of L-selectin from the leading migratory fronts of transmigrating monocytes suggests that this process plays a role in facilitating the directional migration of these cells (2019).

L-selectin is also present on the surface of human embryo trophoblasts prior to implantation into the uterus. Similar to its function in lymphocytes, L-selectin acts as a receptor to facilitate adhesion of the embryo to the site of invasion on the surface epithelium of the uterine endometrium. The embryo secretes human chorionic gonadotropin (hCG), which downregulates anti-adhesion factor, MUC-1, located on the uterine epithelium at the site of invasion. Removal of MUC-1 exposes the oligosaccharide ligands of the uterine epithelium, thus allowing binding by the L-selectin receptor of the trophoblast cell, followed by embryo adhesion and invasion.

vWF-A1. The ability of platelets to tether to and translocate on injured vascular endothelium is supported by the interaction between the platelet glycoprotein receptor Ib alpha (GPIb(alpha)) and the A1 domain of von Willebrand factor (vWF-A1). The kinetics that govern platelet interactions with vWF in hemodynamic flow have been characterized precisely (pubmed.ncbi.nlm.nih.gov/12080112/). The GPIb(alpha)-vWF-A1 tether bond has been shown to display similar kinetic attributes as the selectins including: 1) the requirement for a critical level of hydrodynamic flow to initiate adhesion, 2) short-lived tethering events at sites of vascular injury in vivo, and 3) a fast intrinsic dissociation rate constant. Thus, flow dependent adhesion and rapid and force-dependent kinetic properties are the predominant features of the GPIb(alpha)-vWF-A1 tether bond that in part explains the preferential binding of platelets to vWF at sites of vascular injury, the lack of spontaneous platelet aggregation in circulating blood, and a mechanism to limit thrombus formation. These biophysical properties help to make GPIB(alpha) receptor binding to the Al domain of VWF to be another potentially advantageous type of adhesion molecule for drug targeting, as an example. Note, however, that while neither molecule is believed to be present on the surface of cancer cells, vWF is present in abundance in whole blood and is a macromolecule with various binding sites (e.g, with the ability to bind collagen and unfold) and also stretches under shear stress to expose binding sites.

### D. Purification

In certain embodiments, the deliver particles of the present disclosure may be purified. The term “purified,” as used herein, is intended to refer to a composition, isolatable from other components, wherein the particles is purified to any degree relative to its previous state. Where the term “substantially purified” is used, this designation will refer to a composition in which the particles form the major component of the composition, such as constituting about 50%, about 60%, about 70%, about 80%, about 90%, about 95% or more of the molecules or weight in the composition.

Particle purification techniques are well known to those of skill in the art. These techniques involve, at one level, the crude portioning of the particles. Particles of interest may then be purified using chromatographic and electrophoretic techniques to achieve partial or complete purification (or purification to homogeneity). Analytical methods particularly suited to the preparation of a pure nanoparticle composition are ion-exchange chromatography, exclusion chromatography; polyacrylamide gel electrophoresis; isoelectric focusing. Centrifugation, gel filtration, reverse phase, hydroxylapatite and affinity chromatography; and combinations of such and other techniques.

### III. Conjugation Chemistries

A variety of conjugation methods may be employed to dispose agent on or in a delivery particle. Where antibodies are concerned, several methods are known in the art for such attachment or conjugation. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3cx-6α-diphenylglycouril-3 attached to the antibody (U.S. Pat. Nos. 4,472,509 and 4,938,948). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature (O'Shannessy et al., 1987). This approach has been reported to produce diagnostically and therapeutically promising antibodies which are currently in clinical evaluation.

In some embodiments, the antibody may be conjugated using pClick. The conjugation can occur at the Fc receptor binding site, such as the CH2-CH3 junction of the antibody. The conjugation may employ 4-fluorophenyl carbamate lysine (FPheK). The FPheK can be attached to a fragment of the B domain of protein A (FB protein) from Staphylococcus aureus. In some embodiments, pClick conjugation comprises conjugation of an antibody with an azide functional moiety with a bicyclo[6.1.0]nonyne (BCN) functionalized particle.

In some embodiments, the conjugation method is a site-specific conjugation comprising cysteine chemistry comprising engineered cysteine substitutions at positions on the light and heavy chains that provide reactive thiol groups and do not perturb immunoglobulin folding and assembly or alter antigen binding. Another conjugation method comprises site-specific introduction of aldehyde groups into recombinant proteins using the 6-amino-acid consensus sequence recognized by the formylglycine-generating enzyme (aldehyde tag). The aldehyde tag is no larger than a His6 tag. Yet a further conjugation method comprises remodeled Fc N-glycans of antibodies using mutant glycosyltransferases, such as mutant beta1,4-galactosyltransferase or transglutaminase-mediated site-specific conjugation. In some embodiments, the conjugation method comprises use of disulfide bridges.

### IV. Formulation and Administration

The present disclosure provides pharmaceutical compositions comprising nanoparticles and compositions for generating the same. Such compositions comprise a prophylactically or therapeutically effective amount of nanoparticles and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the antibody or fragment thereof, preferably in purified form, together with a suitable amount of carrier so as to provide the form for proper administration to the patient. The formulation should suit the mode of administration, which can be oral, intravenous, intraarterial, intrabuccal, intranasal, nebulized, bronchial inhalation, intra-rectal, vaginal, topical or delivered by mechanical ventilation.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### V. Combination Therapy

In the context of the present disclosure, it also is contemplated that the functionalized delivery particles described herein could be used similarly in conjunction with additional chemo- or radiotherapeutic intervention, or other treatments. It also may prove effective, in particular, to combine the functionalized delivery particles with other therapies targeting different aspects of cancer biology.

To kill cells, inhibit cell growth, inhibit metastasis, inhibit angiogenesis or otherwise reverse or reduce the malignant phenotype of tumor cells, using the methods and compositions of the present disclosure, one would generally contact a “target” cell with the functionalized delivery particles according to the present disclosure and at least one other agent. These compositions would be provided in a combined amount effective to kill or inhibit proliferation of the cell. This process may involve contacting the cells with the functionalized delivery particles according to the present disclosure and the other agent(s) or factor(s) at the same time. This may be achieved by contacting the cell with a single composition or pharmacological formulation that includes both agents, or by contacting the cell with two distinct compositions or formulations, at the same time, wherein one composition includes the functionalized delivery particles according to the present disclosure and the other includes the other agent.

Alternatively, the functionalized delivery particle therapy may precede or follow the other agent treatment by intervals ranging from minutes to weeks. In embodiments where the other agent and the functionalized delivery particles are applied separately to the cell, one would generally ensure that a significant period of time did not expire between each delivery, such that the agent and expression construct would still be able to exert an advantageously combined effect on the cell. In such instances, it is contemplated that one would contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations. It also is conceivable that more than one administration of the functionalized delivery particles and/or the other agent will be desired. Various combinations may be employed, where the functionalized delivery particles according to the present disclosure therapy is “A” and the other therapy is “B”, as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Administration of the therapeutic agents of the present invention to a patient will follow general protocols for the administration of that particular secondary therapy, taking into account the toxicity, if any, of the antibody treatment. It is expected that the treatment cycles would be repeated as necessary. It also is contemplated that various standard therapies, as well as surgical intervention, may be applied in combination with the described cancer therapies.

The skilled artisan is directed to “Remington's Pharmaceutical Sciences” 15th Edition, Chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Chemotherapy. Cancer therapies also include a variety of combination therapies with both chemical and radiation-based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein transferase inhibitors, transplatinum, 5-fluorouracil, vincristine, vinblastine and methotrexate, Temazolomide (an aqueous form of DTIC), or any analog or derivative variant of the foregoing. The combination of chemotherapy with biological therapy is known as biochemotherapy. The present invention contemplates any chemotherapeutic agent that may be employed or known in the art for treating or preventing cancers.

Radiotherapy. Other factors that cause DNA damage and have been used extensively include what are commonly known as y-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic agent and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

Immunotherapy. Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T-cells and NK cells. The combination of therapeutic modalities, i.e., direct cytotoxic activity and inhibition or reduction of Fortilin would provide therapeutic benefit in the treatment of cancer.

Immunotherapy could also be used as part of a combined therapy. The general approach for combined therapy is discussed below. In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to anticancer effects with immune stimulatory effects. Immune stimulating molecules also exist including cytokines such as IL-2, IL-4, IL-12, GM-CSP, gamma-IFN, chemokines such as MIP-1, MCP-1, IL-8 and growth factors such as FL T3 ligand. Combining immune stimulating molecules, either as proteins or using gene delivery in combination with a tumor suppressor such as mda-7 has been shown to enhance anti-tumor effects (Ju et al., 2000).

As discussed earlier, examples of immunotherapies currently under investigation or in use are immune adjuvants (e.g., Mycobacterium bovis, Plasmodium falciparum, dinitrochlorobenzene and aromatic compounds) (U.S. Pat. Nos. 5,801,005; 5,739,169; Hui and Hashimoto, 1998; Christodoulides et al., 1998), cytokine therapy (e.g., interferons, and; IL-1, GM-CSP and TNF) (Bukowski et al., 1998; Davidson et al., 1998; Hellstrand et al., 1998) gene therapy (e.g., TNF, IL-1, IL-2, p53) (Qin et al., 1998; Austin-Ward and Villaseca, 1998; U.S. Pat. Nos. 5,830,880 and 5,846,945) and monoclonal antibodies (e.g., anti-ganglioside GM2, anti-HER-2, anti-p185) (Pietras et al., 1998; Hanibuchi et al., 1998; U.S. Pat. No. 5,824,311). Herceptin (trastuzumab) is a chimeric (mouse-human) monoclonal antibody that blocks the HER2-neu receptor. It possesses antitumor activity and has been approved for use in the treatment of malignant tumors (Dillman, 1999). Combination therapy of cancer with herceptin and chemotherapy has been shown to be more effective than the individual therapies. Thus, it is contemplated that one or more anti-cancer therapies may be employed with the tumor-associated HLA-restricted peptide therapies described herein.

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989). To achieve this, one would administer to an animal, or human patient, an immunologically effective amount of activated lymphocytes in combination with an adjuvant-incorporated antigenic peptide composition as described herein. The activated lymphocytes will most preferably be the patient's own cells that were earlier isolated from a blood or tumor sample and activated (or “expanded”) in vitro. This form of immunotherapy has produced several cases of regression of melanoma and renal carcinoma, but the percentage of responders was few compared to those who did not respond.

A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

Human monoclonal antibodies are employed in passive immunotherapy, as they produce few or no side effects in the patient. However, their application is somewhat limited by their scarcity and have so far only been administered intralesionally. Human monoclonal antibodies to ganglioside antigens have been administered intralesionally to patients suffering from cutaneous recurrent melanoma (Irie & Morton, 1986). Regression was observed in six out of ten patients, following, daily or weekly, intralesional injections. In another study, moderate success was achieved from intralesional injections of two human monoclonal antibodies (Irie et al., 1989). Possible therapeutic antibodies include anti-TNF, anti-CD25, anti-CD3, anti-CD20, CTLA-4-IG, and anti-CD28.

It may be favorable to administer more than one monoclonal antibody directed against two different antigens or even antibodies with multiple antigen specificity. Treatment protocols also may include administration of lymphokines or other immune enhancers as described by Bajorin et al. (1988). The development of human monoclonal antibodies is described in further detail elsewhere in the specification.

Gene Therapy. In yet another embodiment, the secondary treatment is a gene therapy in which a therapeutic polynucleotide is administered before, after, or at the same time as the tumor-associated HLA-restricted peptide is administered. Delivery of a vector encoding the tumor-associated HLA-restricted peptide in conjunction with a second vector encoding one of the following gene products will have a combined anti-hyperproliferative effect on target tissues. Alternatively, a single vector encoding both genes may be used. A variety of proteins are encompassed within the invention, some of which are described below. Various genes that may be targeted for gene therapy of some form in combination with the present invention are well known to one of ordinary skill in the art and may comprise any gene involved in cancers.

Inducers of Cellular Proliferation. The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally occurring oncogenic growth factor. In one embodiment of the present invention, it is contemplated that anti-sense mRNA directed to a particular inducer of cellular proliferation is used to prevent expression of the inducer of cellular proliferation.

The proteins PMS, ErbA, ErbB and neu are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from protooncogene to oncogene, in one example, results from a valine to glycine mutation at amino acid 12 in the sequence, reducing ras GTPase activity. The proteins Jun, Fos and Myc are proteins that directly exert their effects on nuclear functions as transcription factors.

Inhibitors of Cellular Proliferation. The tumor suppressor oncogenes function to inhibit excessive cellular proliferation. The inactivation of these genes destroys their inhibitory activity, resulting in unregulated proliferation. The most common tumor suppressors are Rb, p53, p21 and pl 6. Other genes that may be employed according to the present invention include APC, DCC, NF-1, NF-2, WT-1, MEN-I, MEN-II, zacl, p73, VHL, C-CAM, MMACI/PTEN, DBCCR-1, FCC, rsk-3, p27, p27/p16 fusions, and p21/p27 fusions.

Regulators of Programmed Cell Death. Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins that share in common structural and sequence homologies. These different family members have been shown to either possess similar functions to Bcl-2 (e.g., BclxL, Bclw, Bcls, Mcl-1, Al, Bfl-1) or counteract Bcl-2 function and promote cell death (e.g., Bax, Bak, Bik, Bim, Bid, Bad, Harakiri).

Surgery. Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### VI. EXAMPLES

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials and Methods

Liposome synthesis, functionalization, and characterization. Multilamellar liposomes were prepared via the thin lipid film method by mixing together L-α-lysophosphatidylcholine (Egg PC, 840051C-200 mg, Avanti Polar Lipids), egg sphingomyelin (Egg SM, 860061C-200 mg, Avanti Polar Lipids), ovine wool cholesterol (700000P-100 mg, Avanti Polar Lipids), 1,2-dioleoyl-sn-glycero-3-[(N-(5-amino-1-carboxypentyl) iminodiacetic acid)succinyl] nickel salt (18:1 DGS-NTA (Ni), 860061C-200 mg, Avanti Polar Lipids), 1,2-distearoyl-sn-glycero-3-phosphoethanolamine-N-[maleimide (polyethylene glycol)-2000] (DSPE-PEG (2000) Maleimide, 880126C-25 mg, Avanti Polar Lipids), and 1,1′-Dioctadecyl-3,3,3′,3′-Tetramethylindo-carbocyanine Perchlorate (DiI, D282, Thermo Fisher). The molar ratios were 47.5%:28%:20%: 2%:2%:0.5% for Egg PC:Egg SM:Cholesterol:DGS-NTA(Ni): DSPE-PEG (2000) Maleimide: DiI (Mitchell et al., 2014; Chandrasekaran et al., 2016). The solution was dried under vacuum overnight and then the lipid cake was rehydrated in 1 mL of HBSS with Ca²⁺ and Mg²⁺ (14025-092, Gibco). Resulting multilamellar liposomes were extruded through membranes with pore sizes first of 400 nm (Nucleopore Track-Etch Membrane 0.4 μm, 800282, Whatman), then 200 nm (Nucleopore Track-Etch Membrane 0.2 μm, 10417004, Whatman), and lastly 100 nm to obtain unilamellar liposomes with a diameter of 100 nm (Nucleopore Track-Etch Membrane 0.1 μm, 800309, Whatman). Filter supports (610014-1EA, Avanti Polar Lipids) were used in each extrusion step to prevent membrane tearing.

To understand the binding of the DA liposome system to different cell types, four different functionalized liposomes were prepared: bare, anti-CSV half-antibody (CSV), ES, anti-CSV half-antibody+ES (DA). Half antibodies were produced by incubating the unconjugated monoclonal human anti-cell-surface-vimentin antibody (H00007431-M08, Abnova) with the reducing agent 2-MEA (153770050, Acros Organics) (Yoshitake et al., 1979). A 2M 2-MEA stock solution was prepared in a reaction buffer containing 150 mM potassium acetate (AM9610, Invitrogen), 10 mM EDTA (E57020-500.0, Research Products International), and 100 mM NaCl (S23020-1000.0, Research Products International). Antibodies were incubated at a volume ratio of 2:8 (mAb to 2-MEA stock solution) for 2 hr at 37° C. Desalting columns (Zeba spin desalting column 7 k MWCO 0.5 mL, 89882, Thermo Fisher Scientific) were used to remove excess 2-MEA in the solution. Liposomes were incubated overnight at 4° C. on a rotator to achieve a final concentration of 6 half-antibodies and 2 recombinant human his-tagged ES (724-ES-100, R&D Systems) on the surface knowing that the starting amount of lipids was 10 μmol and that 100 nm liposomes are made up of 80,047 lipids (Dennison et al., 2009). Liposomes (in 10 μL aliquots) were flash frozen in liquid nitrogen for 15 seconds for long term storage with 10 μmol of D-(+)-trehalose dihydrate (T9531-5G, Sigma Aldrich) per μmol of lipids (Jain & Roy, 2009). Functionalization was confirmed by dynamic light scattering measurements of the liposome diameter on a Malvern Panalytical Advanced Series Ultra Zetasizer.

To study the therapeutic efficacy of this delivery system, liposomes with TRAIL only, TRAIL+anti-CSV half-antibody (t-CSV), TRAIL+ES (t-ES), and TRAIL+anti-CSV half-antibody+ES (t-DA) were prepared. Lipid composition was altered to remove the fluorescent lipid dye Dil to allow for cell death assays using Annexin V and PI. The molar ratios were 48%: 28%:20%:2%:2% for Egg PC:Egg SM:Cholesterol:DGS-NTA(Ni): DSPE-PEG (2000) Maleimide. Liposome synthesis and functionalization was carried out following the steps above. His-tag TRAIL was incubated with the anti-CSV half antibodies and the ES to achieve 10 TRAIL molecules (BML-SE721-0100, Enzo Life Sciences) on the surface of each liposome.

Cell Culture and Reagents. The neutrophil-like cancer cell line PLB985 (ACC-139, DSMZ) and the colorectal cancer cell line HCT116 (CCL-247, ATCC) were utilized throughout this study. PLB985 cells were cultured in RPMI media with L-glutamine (RPMI) supplemented with 10% (v/v) HI FBS (16140-071, Gibco) and 1% PenStrep (15140-122, Gibco). HCT116 cells were cultured in McCoy's media supplemented with 10% (v/v) FBS and 1% PenStrep. Humidified culture conditions were maintained at 37° C. and 5% CO₂ levels. PLB985 cells were washed and resuspended in HBSS with Ca²⁺ and Mg²⁺ at 1M cells per 1 mL. HCT116 cells were detached using trypsin (25200-056, Gibco), then washed and resuspended in HBSS with Ca²⁺ and Mg²⁺ at 1M cells per 1 mL.

Uniform fluid shear stress experiments with a single cell type. Cone-and-plate viscometers (Brookfield) equipped with a CP-40 spindle were used to simulate the FSS that immune cells experience in the circulation (Greenlee et al., 2022; Hope et al., 2021). Before shearing, the cone and plate of the viscometers were blocked with 5% BSA (A1470-100G, Sigma Aldrich) in HBSS with Ca²⁺ and Mg²⁺. Then, 500,000 PLB985 cells were resuspended in 0.5 mL HBSS with Ca²⁺ and Mg²⁺ and placed in the viscometers with 10 μL of liposomes. Uniform FSS was applied to the sample at 188 s⁻¹ for 30 min at room temperature. This is equivalent to 5 dynes/cm² of FSS. Static controls were performed by placing 0.5 mL of the cell solution with the liposomes into centrifuge tubes and placing them on a rocker for 30 min. This is equivalent to less than 0.05 dynes/cm² of FSS. The samples were washed twice in HBSS with Ca²⁺ and Mg²⁺ at 300×g for 5 min, then fixed in 4% PFA (50-980-495, Fisher Scientific) for 15 min. These steps were repeated using the HCT116 cells to test for liposome binding to colorectal cancer cells.

Protocols using human subjects were approved by the Institutional Review Board at Vanderbilt University. After informed consent was given, peripheral blood from healthy volunteers was collected into vacutainer tubes containing sodium citrate buffer (363083, BD). To assess liposome binding to healthy leukocytes, 10 μL of liposomes were added to 2 mL of heathy whole blood and sheared in a Brookfield viscometer using a CP-41 spindle at 188 s⁻¹ for 30 min at room temperature. Static controls were performed by placing 0.5 mL of healthy whole blood with the liposomes into centrifuge tubes and placing them on a rocker for 30 min. Blood separation was performed by gradient centrifugation. Two mL of 1-step polymorphs (AN221725, Accurate Chemical) was warmed to 37° C. in a water bath, and then 2 mL of blood with liposomes was carefully layered on top. This was centrifuged for 47 min at 500×g with acceleration 4 and deceleration 2. The buffy coat was carefully removed and fixed in 4% PFA for 15 min and washed twice by centrifuging at 300×g for 5 min.

Nanoparticle transfer experiments. First, transfer efficacy between the PLB985 cells and the HCT116 cells was assessed using a cone-and-plate viscometer equipped with a CP-40 spindle. Before shearing, the cone and plate of the viscometers were blocked with 5% BSA in HBSS with Ca²⁺ and Mg²⁺. Then, 500 k PLB985 cells were resuspended in 0.5 mL HBSS with Ca²⁺ and Mg²⁺ and placed in the viscometers with 10 μL of liposomes. Uniform FSS was applied to the sample at 188 s⁻¹ for 30 min at room temperature. Samples were washed to remove any unbound liposomes in the suspension via centrifugation at 300×g for 5 min. Viscometers were also rinsed with HBSS with Ca²⁺ and Mg²⁺. The PLB985 cells decorated with liposomes were resuspended in 0.5 mL of HBSS with Ca²⁺ and Mg²⁺ and spiked with 500 k HCT116 cells. The sample was then sheared and additional 2 hrs at 188 s⁻¹. Lastly, the samples were washed twice in HBSS with Ca²⁺ and Mg²⁺ at 300×g for 5 min, then fixed in 4% PFA for 15 min at RT.

Flow cytometry. To test for the presence of cell surface vimentin in all of the cells used throughout the study, 500 k PLB985 and HCT116 cells were fixed in 4% PFA for 15 min and then blocked in 5% BSA for 30 min. Cells were stained with either a monoclonal human anti-CSV antibody tagged with FITC (H00007431-MF08, Abnova) or a mouse IgG2b kappa isotype antibody tagged with FITC (400310, BioLegend) at a 3:100 ratio in 5% BSA for 1 hr. CSV expression on the surface on healthy leukocytes before and after shear stress exposure was also investigated. Fresh whole blood (2 mL) was either sheared in a viscometer at 188 s⁻¹ with a CP-41 spindle or incubated in a centrifuge tube on a rocker for 2 hrs. Blood was separated, fixed, and stained following the protocols detailed above. Samples were run through a Guava EasyCyte 12HT flow cytometer using the Green-B laser. Flow cytometry data was analyzed on FlowJo.

Binding of liposomes to PLB985 cells, HCT116 cells, and healthy leukocytes was confirmed by running the samples through the flow cytometer using the Red-B laser. Granulocyte and monocyte+lymphocyte populations were gated using the forward scatter and side scatter plot (Leach et al., 2013). In the transfer experiments from the PLB985 to the HCT116 cells, the PLB985 cells were stained with DAPI at a 1:1000 ratio in HBSS with Ca²⁺ and Mg²⁺ and then washed twice by centrifuging at 300×g for 5 min before placing the samples in the flow cytometer. For the transfer experiments in whole blood spiked with HCT116 cells, the leukocytes were stained with an anti-CD45 antibody tagged with eFluor 450 (48-0459-42, Invitrogen) at 3:100 in 5% BSA for 1 hr after the magnetic column separation. Samples for the transfer experiments were run through the flow cytometer using the Red-B to identify liposome coated cancer cells and Blue-V laser to gate out the PLB985 cells (DAPI) and the leukocytes (CD45 stain).

Confocal microscopy. For microscopy, cells were stained with DAPI (1.5:1000) for 30 min and visualized on a Zeiss LSM900 inverted laser scanning microscope equipped with a 63x objective with oil immersion.

Statistical analysis. Where appropriate, unpaired, non-parametric t-tests, ordinary one-way ANOVAs with Tukey multiple comparisons test, and two-way ANOVAs with Šídák's multiple comparisons test were used. Significance was shown as *p<0.05, **p<0.01, ***p<0.001, and ****p<0.0001. A sample size of n=3 was used. All values are presented as mean±SD. All statistical analysis and plot preparation was performed on GraphPad Prism.

### Example 2—Results

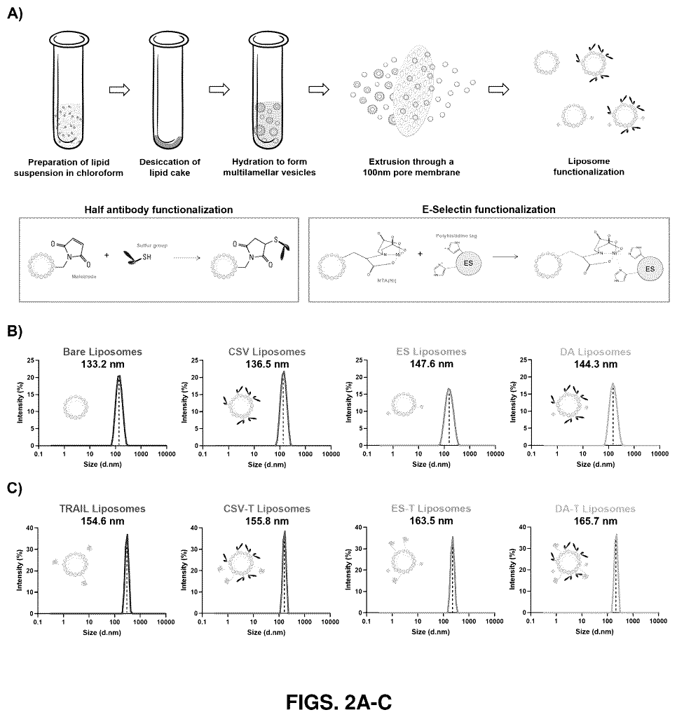

In this study, the inventors prepared liposomes which can adhere reversibly to healthy leukocytes and strongly to CTCs in the circulation. The goal of this design is to have the liposomes circulate loosely bound to healthy leukocytes as they travel in the bloodstream. Then, when the leukocyte collides with a CTC, the liposomes attach irreversibly to the cancer cell. When the collision is over, the liposome will have transferred onto the surface of the CTC, effectively delivering the TRAIL therapeutic cargo without harming the leukocyte and evading filtration by the kidneys. Liposomes were prepared via the lipid cake and extrusion technique following the step in FIG. 2A. The lipids suspended in chloroform were mixed in a glass vial. After overnight desiccation to remove the solvent, the lipids were rehydrated in buffer. The resulting multilamellar lipid particles were extruded through a membrane with 100 nm pores to obtain the desired liposome size. Functionalization was performed using click-chemistry. His-tagged E-selectin and His-tagged TRAIL readily bind to NTA(Ni) groups. Free sulfur groups on cleaved half-antibodies easily bind to maleimide.

Dual affinity liposomes were obtained by incubating with both the his-tagged E-selectin and the anti-cell surface vimentin (CSV) half antibodies. Three control liposomes were synthesized: liposomes with no surface modalities (bare liposomes), liposomes with only the CSV half-antibodies (CSV liposomes), and liposomes with only E-selectin on the surface (ES liposomes). To understand the therapeutic efficacy of the liposomal system, these four liposome groups were synthesized and his-tagged TRAIL was also added to their surface. Functionalization of the liposomes was confirmed by measuring the liposome size via dynamic light scatterings. Bare liposomes had a mean dynamic radius of 133.2 nm (FIG. 2B). Adding the half-antibodies and the E-selectin added 3 nm and 14 nm to the dynamic diameter, respectively. When both targeting moieties were present on the liposome surface, the diameter was 144.3 nm. Adding TRAIL increased all liposome dynamic diameters by 10 nm on average (FIG. 1C).

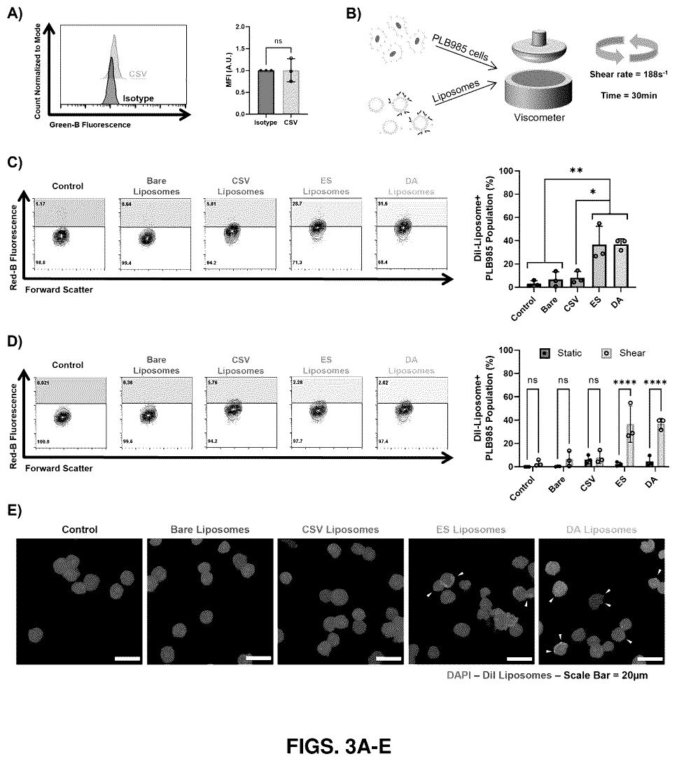

To investigate the transfer efficacy of the dual affinity nanoparticles from healthy leukocytes to cancer cells in the circulation, the neutrophil-like cell line PLB985 and the colorectal cancer cell line HCT116 were used. PLB985 cells were investigated for CSV expression, to confirm that they would appropriately model the attachment of liposomes through only the E-selectin ligands. These cells did not significantly express CSV, confirming that they would be an appropriate cell line model for the transfer experiment (FIG. 3A). The binding capacity of the liposomes to the neutrophil model cell line PLB985 was tested under physiologically relevant FSS conditions. The cells were incubated with the nanoparticles in a cone-and-plate viscometer for 30 min (FIG. 3B).

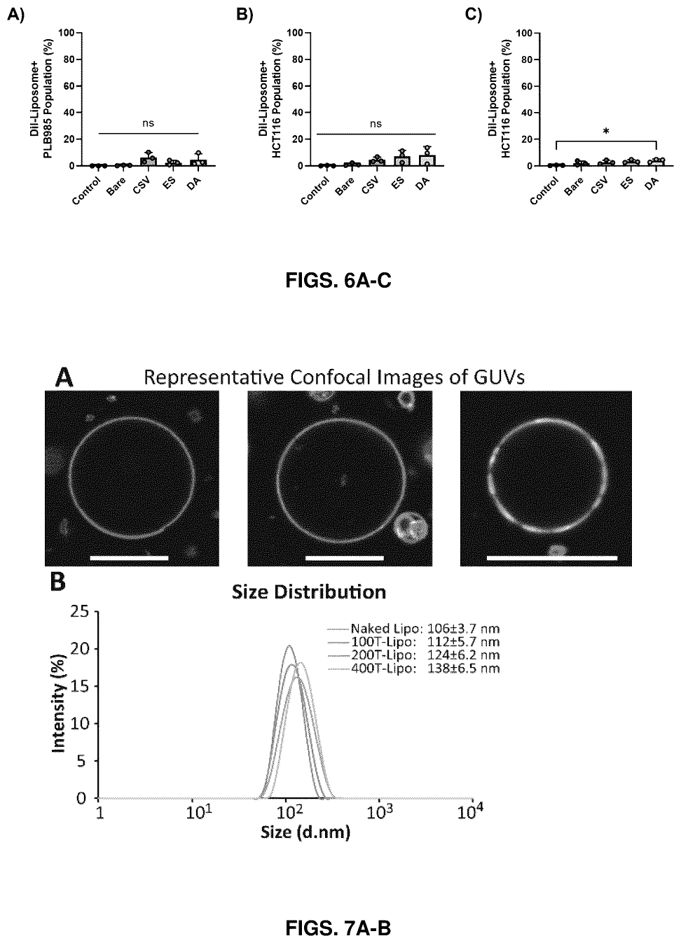

The percentage of cells decorated with liposomes was quantified using flow cytometry. Under FSS conditions, 36% of the cells in both the ES liposome group and the DA liposome group were decorated (FIG. 3C). This was a 6-fold increase compared to the bare liposome group. No significant levels of binding were observed for the bare liposomes and the CSV liposomes compared to the control where no liposomes were present in the sample. When the experiment was repeated with static conditions, the percentage of cells decorated with liposomes decreased significantly by 15-fold and 8-fold in the ES and DA groups, respectively (FIG. 3D). No significant changes in binding were observed between each of the liposome groups under static conditions (FIG. 6A). PLB985 cells decorated with liposomes under FSS were observed using confocal microscopy (FIG. 3E). In the micrographs, liposomes can be seen surrounding the cells in the ES and DA liposome conditions.

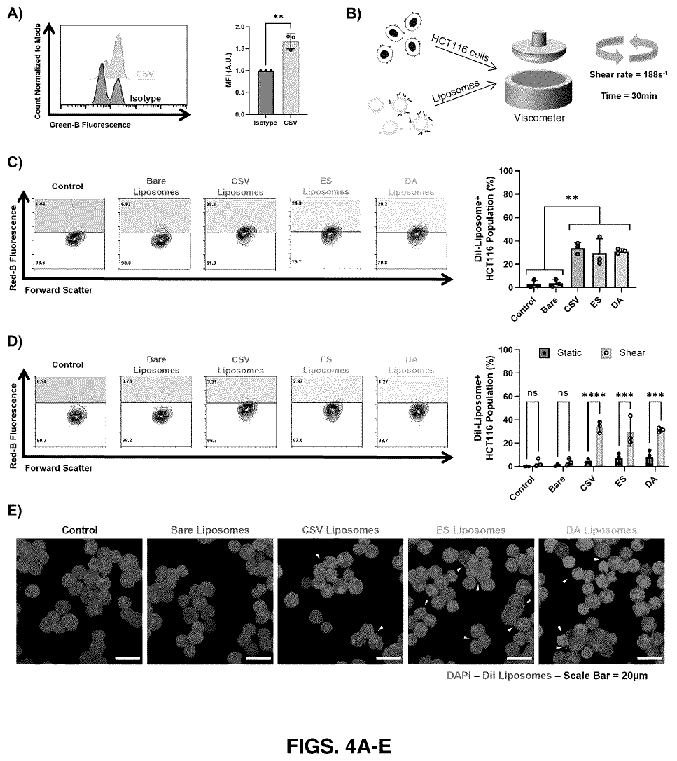

To confirm that the DA liposomes attached to cancer cells, the HCT116 cells were sheared in a cone-and-plate viscometer with the liposomes. First, the expression of CSV on the HCT116 cells was quantified via flow cytometry (FIG. 4A). There was significant expression of CSV shown by a 67% increase in MFI compared to the isotype antibody control. The cells were sheared in a cone-and-plate viscometer with each of the liposome types for 30 min at a FSS of 5dynes/cm² (FIG. 4B). Flow cytometry analysis showed an 8-fold increase in the percentage of HCT116 cells decorated with the CSV, ES and DA liposomes compared to the bare liposome group (FIG. 4C). There was no significant change in binding between the bare liposome group and the control without liposomes. When the liposomes were incubated with the HCT116 cells under static conditions, there was a significant decrease in binding to the CSV, ES, and DA liposomes (FIG. 4D). No significant changes in binding were observed between each of the liposome groups under static conditions (FIG. 6B). The HCT116 cells decorated with liposomes under FSS were observed using confocal microscopy (FIG. 4E). The signal in the red channel (DiI+ liposomes) was especially visible in the cells treated with the CSV, ES, and DA liposomes. Liposomes were localized to the outside of the cells.

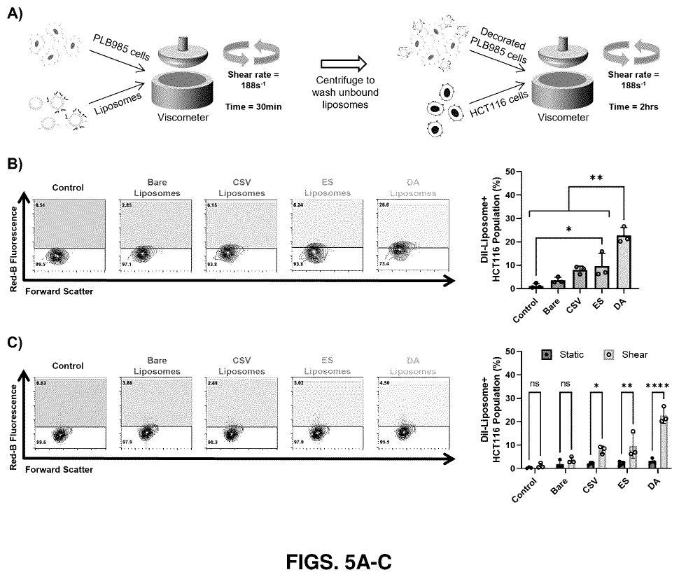

Liposome transfer was tested between neutrophil-like cell line PLB985 and the HCT116 colorectal cancer cells. First, the liposomes were incubated with the PLB985 cells for 30 mins (FIG. 5A). The cells suspension was washed to remove any unbound liposomes from the solution. Then the PLB985 cells decorated with liposomes were spiked with the HCT116 cells and sheared in the viscometers for 2 hours. The DA liposomes had a significantly higher transfer efficacy than all other liposomes formulations (FIG. 5B). The percentage of HCT116 cells decorated with DA liposomes was 23%, this was a 3-fold increase compared to the percentage of decorated HCT116 cells incubated with the ES liposomes. Interestingly, the ES liposomes did transfer under shear conditions, but had a low efficacy in doing so only transferring to 10% of the HCT116 cell population. As expected, when the transfer efficacy was tested in static conditions, there was a significant decrease in the transfer of the CSV, ES and DA liposomes (FIG. 5C). In static conditions, there was a very small but significant increase in the transfer efficacy of the DA liposomes, but the average percentage of cells that the DA liposomes transferred onto was only 3.3% (FIG. 6C).

Phase separation and liposome tuning. Phase separation naturally exists in biological membranes and is directly connected to various events in the life of a living cell, including virus entry and exit, sorting of proteins to various cellular locations, membrane fusion and fission, and transmission of signals. Giant unilamellar vesicles (GUVs), artificially made enclosed lipid bilayer membrane structures at micrometer scales, are commonly used as a model to study membrane properties and structure. To mimic the phase separation behavior, a 3-component lipid mixture such as DSPC/DOPC/cholesterol is often chosen for the preparation of GUVs. In the mixture, the two least miscible glycerophopholipids, with saturated and unsaturated acyl chain respectively, form a liquid ordered (Lₒ) or gel phase (when cholesterol is excluded), and a liquid disordered (L_{α}) phase respectively. In recent years, the nanoscale counterpart of phase-separated GUVs, phase-separated liposomes, are emerging for the study of membrane properties and structure at nanometer scale, including the spatial distribution of membrane-associated proteins. The inventors' lab uses phase-separated liposomes for the delivery of therapeutic proteins to treat cancer and other diseases.

TRAIL activates the extrinsic apoptotic pathway upon binding to its cognate death receptors 4 and 5 (DR4 and DR5, also known as TRAIL-R1 and TRAIL-R2, respectively). Like other members of the TNF family, TRAIL must form a homotrimer to achieve its cancer cell-specific toxicity. DR4/5 receptors bind to the gaps between two neighboring monomers in the TRAIL trimer, one receptor to each of the three gaps. The binding results in receptor trimerization that leads to conformational changes in the TRAIL receptors. However, this conformation change alone is not always sufficient to transduce the intracellular signal. It seems that further multimerization of the receptors is required to complete the apoptotic signaling cascade. Previous studies have shown that some DRs need to be further cross-linked or oligomerized in high-order clusters on the cell surface to correctly induce the intracellular signal. It is possible that death ligands like TRAIL may achieve this by clustering into supra-molecular structures on the membranes of cells or cell-secreted vesicles. This idea is supported by the discovery that TRAIL is much more potent as a naturally expressed transmembrane protein than when in its free, soluble form.

The inventors recently reported that TRAIL can be concentrated to the smaller domain of phase-separated liposomes to increase cancer specific killing capacity by adjusting its distribution density (Zhang & King, 2024). They first prepared GUVs using the same lipid formulation as that of the liposomes to confirm that the lipid bilayer membrane structures do exhibit phase separation. They were able to conjugate TRAIL proteins only to the smaller DOPC domains as the lipids for conjugation preferably stay in the same domain. The number of TRAIL proteins was varied to change the density of TRAIL on liposome surface (FIGS. 7A-B). In theory, it is possible to fully or nearly fully coat a homogenous liposome with TRAIL to promote the formation of TRAIL clusters. But this could compromise the stability of liposomes in vivo and the long-term stability in vitro.

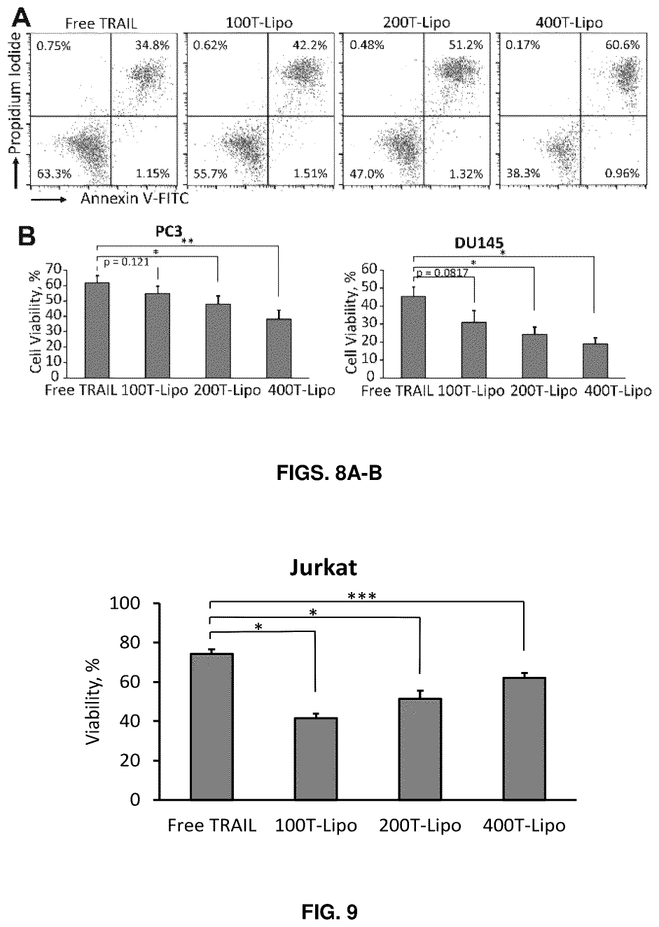

The inventors used human prostate cancer PC3 and DU145 cells which exhibit high and medium sensitivity to TRAIL, respectively, to compare the apoptotic activity of the phase-separated liposomes conjugated with different numbers of TRAIL molecules. The TRAIL liposomes showed higher cytotoxic effects in PC3 and DU145 cells than free TRAIL after 24 h of treatment. More importantly, the liposomes conjugated with more TRAIL molecules were found more potent than those with fewer TRAIL molecules at the same total concentration of TRAIL (FIGS. 8A-B).

In the treatment of human T lymphocyte immortalized Jurkat cells which are known to be highly sensitive to TRAIL-induced apoptosis, however, the TRAIL liposomes with fewer TRAIL were found more potent than those with more TRAIL, although all three formulations are more apoptotic than free TRAIL (FIG. 9). It is likely that the distribution of TRAIL in the three formulations does not match that of the death receptors on the surface of Jurkat cells as in the case of PC3 and DU145 cells.

These results support the idea that the cancer-specific killing capacity of liposomal TRAIL can be adjusted by changing the number of TRAIL conjugated to the smaller phase of phase-separated liposomes. The inventors have thus provided a facile method to prepare an optimal liposome formulation of TRAIL for a specific cell line.

### Example 3—Discussion

In this study, the inventors successfully designed and validated a nanoparticle system which can transfer between a carrier cell and a target cell under physiologically relevant FSS conditions found in the circulation (FIG. 1). Liposomal nanoparticles were decorated with E-selectin and an anti-cell surface vimentin half antibody (FIG. 2A). E-selectin was chosen as it is expressed in healthy adult granulocytes and the ES-ESL bond is easily ruptured, providing an ideal tethering mechanism to the carrier cells. An antibody was used to target and non-reversibly adhere to the target cell as these bonds require very high forces to rupture. The liposomes were designed to carry the cancer immunotherapy TRAIL. This ligand selectively kills cancer cells by inducing apoptosis when it binds to death receptors (DR) 4 and 5 (Greenlee et al., 2021; Lemke et al., 2014; Hope et al., 2019). These are found on the cell surface, and exclusively expressed by cancer cells.

This nanoparticle system provides a platform for an easily customizable targeted delivery mechanism to CTCs. As a proof of concept, cell surface vimentin was chosen as the target for the cancer cells as it is selectively expressed on their surface and not on that of healthy cells (Satelli & Li, 2011; Satelli et al., 2017; Satelli & Li, 2011). Although, many other markers for CTCs are explored in the literature for targeted delivery of therapeutics in the circulation. These include but are not limited to CD44, CD133, EpCAM, PSMA, and more (Lourenço et al., 2021; Faltas, 2012; Marshall & King, 2015; Lin et al., 2018). The technology could additionally be adapted to specific patient CTC marker expressions to make it a customizable and finely targeted treatment by adding half-antibodies against different overexpressed surface proteins. To confirm that an anti-CSV antibody would be an appropriate targeting moiety for the colorectal cancer cell line HCT116, these cells were validated via flow cytometry (FIG. 4A).

For the liposomes surface functionalization chemistry, reactions that could be performed via click-chemistry were chosen. E-selectin with a poly-histidine tag was attached to the liposome surface by incorporating a lipid with NTA(Ni) into the formulation. This is also how the his-tagged TRAIL was conjugated. Additionally, a lipid with a maleimide group was used to synthesize the liposomes, which readily binds to the sulfur group of a cleaved antibody (Lourenço et al., 2021). Changes in the diameter of the liposome indicated successful conjugation of the moieties of the surface (FIGS. 2B-C).

Initial experiments for the transfer efficacy of the DA liposomes will be performed using PLB-985 in place of whole blood. This is a cell line derived from HL-60 which act as a model for human neutrophils, as these are the blood cells with the highest levels of ESL (Munro et al., 1992; Hauert et al., 2002; Millius & Weiner, 2010; Li et al., 2012; Stroud et al., 1996; Huang et al., 2004). In model experiments 5×10⁵ cells/mL concentration was used as it is, on average, the density of polymorphonuclear cells/mL that are found in healthy human blood (Kuhns et al., 2015). Binding capacity of the DA liposomes under FSS conditions to the PLB985 cells was confirmed via flow cytometry and confocal microscopy (FIG. 3C and FIG. 3E). Additionally, there was no significant difference in binding between the ES liposomes and the DA liposomes, showing that the addition of the half-antibodies did not negatively impact the nanoparticle bonding. Under static conditions no liposomes adhered to the PLB985 cells, highlighting the importance of FSS on the ES-ESL bond (FIG. 3D) (Helms et al., 2016; Snook & Guilford, 2010).

Significant binding of the DA liposomes after FSS was observed on the surface of the HCT116 colorectal cancer cells (FIG. 4C and FIG. 4E). The inventors confirmed that there was no significant difference in the DA liposomes binding to the surface of the HCT116 cells compared to the DA liposomes. Again, showing that the addition of different targeting moieties did not affect the binding capacity of the DA liposomes. Interestingly, the ES liposomes bound to the HCT116 cells to the same degree as the CSV liposomes. Healthy leukocytes are not the only cells expressing Sialyl Lewis^{X} ligands. Many cancers do express E-selectin ligands, which play a role in rolling/adhesion to prepare CTCs for extravasation, promoting migration, shear resistance, and survival against immune cells in the circulation (Yasmin-Karim et al., 2014; Kang et al., 2016; Borsig, 2018).

Phase separation in the membrane of liposomes also offers an opportunity to distribute E-selectin and the cancer cell-specific antibody separately in the two distinct phases so that one can control their relative affinity to cells in a more rational manner. The transferring rate of the liposomes can then be changed for efficient killing of cancer cells. Technically, the affinity of the dual affinity liposomes to cell surfaces is partly determined by the total number of E-selectin and antibodies conjugated on the liposome surfaces in addition to the affinity of E-selectin and antibody to their ligand and antigen respectively. The number of E-selectin and antibodies on a liposome can be well controlled because they are conjugated to the liposome surface via his-tag/DGS-NTA (Ni²⁺) and thiol-maleimide respectively, which is biorthogonal. To conjugate the two proteins separately in different phases of liposomes, the two proteins can be incubated with pre-formed phase-separated liposomes either simultaneously or sequentially for efficient conjugation. Other biorthogonal chemistry such as click chemistry may be utilized for the conjugation of the two proteins.

Phase separation could also help with more complete transfer of TRAIL from leukocytes to cancer cells. For homogenous liposomes, the three proteins are randomly distributed among the whole liposome surface. When the liposomes are transferred, it is likely fission of liposomes will occur due to the stretching from the types of cells, resulting in TRAIL partly left with E-selectin-rich liposome patches on the surface of leukocyte. In phase-separated liposomes, TRAIL can be conjugated with the same phases as cancer-specific antibody to facilitate the transfer of TRAIL since the antibody-conjugated liposome patches will be mostly transferred to cancer cells due to their higher affinity.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VII. REFERENCES

The following references, to the extent that they provide exemplary procedural or other details supplementary to those set forth herein, are specifically incorporated herein by reference.
Abdalla et al., PLOS ONE 16, e0252897 (2021).
Allen et al., Biochemistry 36, 7457-7463 (1997).
Alieva et al., Clin Exp Metastasis 35, 319-331 (2018).
Borsig, Glycobiology 28, 648-655 (2018).
Butler & Gullino, Cancer Res 35, 512-516 (1975).
Chandrasekaran et al., Biomaterials 77, 66-76 (2016).
Chinen et al., Chemical Reviews 115, 10530-10574 (2015)
Dammer et al., Biophysical Journal 70, 2437-2441 (1996).
Dennison et al., J Virol 83, 10211-10223 (2009).
Dogget et al., Biophysical J. 83(1) 194-205 (2002).
Du et al., Technol Cancer Res Treat 19, 1533033820957005. (2020).
Faltas, Frontiers in Oncology 2 (2012).
Greenlee et al., eLife 10, e67750 (2021).
Greenlee et al., Molecules 27, 5430 (2022).
Gu et al., Nano Today 2, 14-21 (2007).
Harris et al., BMC Cancer 18, 718 (2018).
Hauert et al., The International Journal of Biochemistry & Cell Biology 34, 838-854 (2002).
Helms et al., FEBS Letters 590, 1955-1971 (2016).
Hope et al., Journal of Cell Science 134 (2021).
Hope et al., Cell Death & Disease 10, 837 (2019).
Huang et al., Journal of Biological Chemistry 279, 44915-44923 (2004).
Jain & Roy, Protein Sci 18, 24-36 (2009).
Jyotsana et al., Science Advances 5, (2019).
Kang et al., BMC Cancer 16, 331 (2016).
Karagiannis et al., Sci Transl Med 9, eaan0026 (2017).
King et al., Biophysical J., 88(3), 1676-83 (2005).
Kuhns et al., Curr Protoc Immunol 111, 7.23.1-7.23.16 (2015).
Leach et al., Practical Flow Cytometry in Haematology Diagnosis (John Wiley & Sons, Incorporated) (2013).
Lemke et al., Cell Death Differ 21, 1350-1364 (2014).
Li et al., Experimental Cell Research 318, 1649-1658 (2012).
Lin et al., Annual Review of Biomedical Engineering 20, 329-352 (2018).
Liu et al., Oncology Reports 43, 1525-1535 (2020).
Long et al., Annals of Biomedical Engineering 29, 935-946 (2001).
Longmire et al., Nanomedicine (Lond) 3, 703-717 (2008).
Lourenço et al., Nanomaterials 11, 295 (2021).
Millius & Weiner, Methods Mol Biol 591, 147-158 (2010).
Mitchell et al., Proceedings of the National Academy of Sciences 111, 930-935 (2014).
Mitchell & King, New Journal of Physics 15, 015008 (2013).
Mitchell & King, Expert Review of Anticancer Therapy 14, 641-644 (2014).
Marshall & King, Cel. Mol. Bioeng. 8, 527-529 (2015).
Marshall & King, Translational Cancer Research 5 (2016).
Munro et al., Am J Pathol 141, 1397-1408 (1992).
Naito et al., Journal of Thoracic Oncology 7, 512-519 (2012).
Ortiz-Otero et al., Adv Exp Med Biol 1092, 235-251 (2018).
Ortiz-Otero et al., BMC Cancer 20, 873 (2020a).
Ortiz-Otero et al., Nanoscale Adv. 2, 3942-3953 (2020b).
Ortiz-Otero et al., BMC Cancer 21, 898 (2021).
Perea Paizal et al., Br J Cancer 124, 58-65 (2021).
Poon et al., ACS Nano 13, 5785-5798 (2019).
Rocheleau et al., BMC Struct Biol 16, 10 (2016).
Sastre et al., Oncologist 17, 947-955 (2012).
Satelli & Li, Cellular and Molecular Life Sciences 68, 3033-3046 (2011).
Satelli et al., Oncotarget 8 (2017).
Sawabata et al., Surg Today 46, 1402-1409 (2016).
Schuster et al., Trends in Cancer 7, 1020-1032 (2021).
Shishido et al., Journal of Translational Medicine 17, 294 (2019).
Snook & Guilford, Cell Mol Bioeng 3, 128-138 (2010).
Stroud et al., Biochemistry 35, 770-778 (1996).
Tsai et al., JCO 37, 485-485 (2019).
Tees et al., Biophys J. 2001 February; 80(2): 668-82 (2012).
von Karstedt et al., Nat Rev Cancer 17, 352-366 (2017).
Wayne et al., Journal of Controlled Release 223, 215-223 (2016).
Wild et al., Journal of Biological Chemistry 276, 31602-31612 (2001).
Yasmin-Karim et al., Oncotarget 5, 12097-12110 (2014).
Yao et al., Frontiers in Molecular Biosciences 7, 193 (2020).
Yoshida et al., Surg Today 23, 979-983 (1993).
Yoshitake et al., Eur J Biochem 101, 395-399 (1979).
Zhang & King, Nanoscale Adv., 6, 402-405 (2024).

1. A delivery particle comprising:
(a) an anti-cancer agent;
(b) a first targeting agent that binds to a non-cancer cell surface moiety; and
(c) a second targeting agent that binds to a cancer cell surface moiety,
wherein the relative affinity of said first targeting agent to said second targeting agent is such that the second targeting agent binding with the cancer cell surface moiety will outcompete the first agent binding with the non-cancer cell surface moiety.

2. The delivery particle of claim 1, wherein the relative affinities for said first targeting agent and said second targeting agent are less than 100 pN in strength and at least 1000 pN in strength, respectively, such as about 30 pN and about 1000-2000 pN, respectively.

3. The delivery particle of claim 1, wherein said delivery particle is a bead, a gold particle, a polymeric particle, a vesicle (e.g., nanovesicle), a liposome, a nanoparticle (e.g., lipid nanoparticle), a nanotube, a nanorod, a micelle, or a dendritic macromolecule, such as a lipid nanoparticle comprising phosphatidyl choline, sphingomyelin and cholesterol.

4. The delivery particle of claim 1, wherein the second targeting agent is an antibody to said cancer cell surface moiety.

5. The delivery particle of claim 1, wherein the cancer cell surface moiety is vimentin or PSMA.

6. The delivery particle of claim 1, wherein the first targeting agent is an antibody or receptor for said non-cancer cell surface moiety, such as a receptor for a moiety found on a white blood cell, e.g., wherein the receptor for said non-cancer cell surface moiety is E-selectin, P-selectin, L-selectin or vWF-A1.

7. The delivery particle of claim 1, wherein the anti-cancer agent is located in an internal phase of said delivery particle, such as a chemotherapeutic agent, a radiotherapeutic agent or a toxin.

8. The delivery particle of claim 1, wherein the anti-cancer agent is located on the surface of said delivery particle.

9. The delivery particle of claim 1, wherein the anti-cancer agent is TRAIL or Fas ligand.

10. The delivery particle of claim 1, wherein the first targeting agent is E-selectin, the second targeting agent is an antibody that binds to vimentin and the anti-cancer agent is TRAIL.

11. The delivery particle of claim 1, wherein one or more of said anti-cancer agent, said first targeting agent and said second targeting agent are conjugated to the surface of said delivery particle using click chemistry.

12. The delivery particle of claim 1, wherein the delivery particle is a phase separated liposome comprising at least three lipid types.

13. The delivery particle of claim 12, wherein the anti-cancer agent is linked to one of said at least three lipid types, and/or said first and/or second targeting agent is linked to one of said at least three lipid types.

14. The delivery particle of claim 1, wherein the cancer cell is a circulating cancer cell.

15. The delivery particle of claim 1, wherein the non-cancer cell is a circulating non-cancer cell.

16. The delivery particle according to claim 1, disposed in a pharmaceutically acceptable carrier or diluent.

17. A delivery particle comprising:
(a) a detectable label;
(b) a first targeting agent that binds to a non-cancer cell surface moiety in a reversible fashion; and
(c) a second targeting agent that irreversibly binds to a cancer cell surface moiety.

18-19. (canceled)

20. A method of targeting a circulating cancer cell in a subject comprising administering to said subject a delivery particle according to claim 1.

21. The method of claim 20, wherein the subject has cancer, wherein said delivery particle comprises an anti-cancer agent.

22-29. (canceled)

30. A delivery particle comprising:
(a) a first targeting agent that binds to a first cell; and
(b) a second targeting agent that binds to a second cell,
wherein the relative affinity of said first targeting agent to said second targeting agent is such that the second targeting agent binding to the second cell surface moiety will outcompete the first agent binding to the first cell surface moiety, resulting in transfer of the delivery particle from the first cell to the second cell, within the dynamic environment of blood flow.
